(19) United States
(12) Patent Application Publication
(10) Pub. No.: US 2014/0308301 A1
(43) Pub. Date: Oct. 16, 2014

(54) CD44 MONOCLONAL ANTIBODY FOR THE TREATMENT OF B-CELL CHRONIC LYMPHOCYTIC LEUKEMIA AND OTHER HEMATOLOGICAL MALIGNANCIES

(71) Applicant: The Regents of the University of California, Oakland, CA (US)

(72) Inventors: Thomas J. Kipps, San Diego, CA (US); Suping Zhang, San Diego, CA (US); Christina C. Wu, Escondido, CA (US)

(21) Appl. No.: 14/354,472

(22) PCT Filed: Oct. 26, 2012
(86) PCT No.: PCT/US12/62266

§ 371 Date: Apr. 25, 2014

Related U.S. Application Data
(60) Provisional application No. 61/551,852, filed on Oct. 26, 2011.

(51) Int. Cl. C07K 16/28; G01N 33/574

(52) U.S. Cl. CPC C07K 16/2884; G01N 33/57426; G01N 33/57492
USPC 424/174.1; 530/389.7; 530/387.3; 536/23.53; 435/320.1; 435/69.6; 435/7.23; 435/375

(57) ABSTRACT

Compositions including an antibody specific for CD44 are provided. These antibodies specifically bind to hematologic malignant cells. Methods to use the CD44 antibodies to target cells expressing CD44 for therapeutic and diagnostic purposes are also provided.

### RELATED APPLICATIONS

This application claims the benefit of U.S. Provisional Application No. 61/551,852 filed Oct. 26, 2011, the contents of which are incorporated herein by reference in their entirety.

### GRANT INFORMATION

This invention was made with government support under National Institutes of Health Grant Nos. P01 CA081534 and R37 CA049780. The government has certain rights in the invention.

### BACKGROUND OF THE INVENTION

1. Field of the Invention

The present invention relates generally to antibodies which target hematological malignancies. The invention further relates to antibodies which specifically bind to CD44 and target chronic lymphocytic leukemia cells.

2. Background Information

Hematological malignancies affect blood, bone marrow, and lymph nodes. These malignancies typically derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

B-cell chronic lymphocytic leukemia (CLL), has a highly variable clinical course and is characterized by the clonal expression of CD5+ B cells in blood, secondary lymph tissues and marrow. CLL is a heterologous disease with variable prognosis; some patients have an indolent course and a virtually normal life expectancy, others have aggressive disease and a short survival. Patients with CLL are generally not treated in early stage disease and are monitored for disease progression. Treatment usually starts when the patients quality of life is affected. Although there is no cure for CLL, the disease is typically treatable and current standard chemotherapy regimens have been shown to prolong survival. However, there is a population of CLL patients which is refractory or whom become refractory to the standard chemotherapy regimens.

CLL cell survival is supported by cells within the tissue environment and by signals from the extracellular matrix and interactions with CD44, which is expressed at high levels on CLL cells. CD44 is a multi-structural glycoprotein involved in many physiological and pathological functions, including cell-cell and cell-matrix adhesion, support of cell migration, presentation of growth factors, chemokines or enzymes to corresponding cell surface receptors or relevant substrates, as well as transmission of signals from the membrane to the cytoskeleton or nucleus [Naor, D., et al. Adv. Cancer Res. 71, 241-319, (1997); Lesley, J., et al. Adv. Immunol. 54, 271-335, (1993)]. This protein participates in a wide variety of cellular functions including lymphocyte activation, recirculation and homing, hematopoiesis, and tumor metastasis. This glycoprotein is known to bind to multiple ligands (e.g. fibrinogen, fibronectin, alanine, collagen), the principal one being hyaluronic acid (HA).

While many CLL patients respond to standard chemotherapy regimens for CLL, as noted previously, there is a population of CLL patients which is refractory or whom become refractory to these standard treatments. For this population of patients none of the existing chemotherapy regimens is successful thereby demonstrating a need for new therapies to treat CLL. This invention provides such a therapy, a novel CD44 antibody which is specific for CLL cells.

### SUMMARY OF THE INVENTION

The present invention is based on the seminal generation of an anti-CD44 antibody. Additionally, the invention is based on methods of treatment or prevention for hematological malignancies using an anti-CD44 antibody. Further, this invention provides methods of making a CD44 antibody.

In one aspect, the present invention provides an antibody or antibody fragment which specifically binds CD44.

In another aspect, the antibody fragment includes a Fab fragment, a F(ab)2 fragment, an FV fragment, a single chain FV (scFV) fragment, a dsFV fragment, a CH fragment or a dimeric scFV.

In various embodiments, the antibody or antibody fragment is humanized.

In a further aspect, the invention provides an antibody or antibody fragment which specifically binds CD4 on CLL cells.

In another aspect, the present invention provides an isolated nucleic acid encoding the antibody or antibody fragment of the invention. In a further embodiment, the invention provides an expression vector which contains the nucleic acid encoding the antibody.

In another aspect, the present invention provides a pharmaceutical composition including the antibody or antibody fragment of the invention and optionally a pharmaceutically acceptable carrier.

In another aspect, the present invention provides a method of producing an antibody. The method includes transforming a host cell with an expression construct including a nucleic acid molecule encoding an antibody and culturing the host cell under conditions suitable for producing the antibody, thereby producing the antibody.

In another aspect, the present invention provides a method for detecting CD44 protein in a sample.

In another aspect, the present invention provides a method of targeting an antibody to a cell having an CD44 receptor. The method includes contacting the cell with an antibody of the invention.

In another aspect, the present invention provides a kit to detect the presence of CD44 protein in a sample from a subject that is known or suspected to contain hematological malignant cells. The kit includes the an antibody and instructions for its use in an assay environment.

In another aspect, the present invention provides a method for treating a hematological malignancy in a human subject using an antibody of the invention.

In another aspect, the present invention provides a method for treating or preventing CLL in which an antibody binding to CD44 on CLL cells confers a survival advantage thereon by administering a CD44 antibody of the invention.

In another aspect, the present invention provides a method of monitoring a therapeutic regimen for treating a subject having or at risk of having an hematological malignancy using an antibody of the invention.

In one other aspect, the present invention provides a method for treating or preventing a hematological malignancy in a subject, the method comprising administering to a subject in need thereof a therapeutically effective amount an antibody to CD44, wherein the hematological malignancy is refractory to chemotherapy and/or biotherapy. In one embodiment, the chemotherapy comprises a purine nucleoside analog and/or an alkylating agent. In another embodiment, the hematological malignancy is refractory to chemotherapy and biotherapy. In an additional embodiment, the biotherapy comprises a monoclonal antibody. In one embodiment, the monoclonal antibody is an anti-CD20 antibody. In some embodiments, the hematological malignancy is a leukemia. In another embodiment, the leukemia is lymphocytic leukemia. In one other embodiment, the lymphocytic leukemia is B-cell chronic lymphocytic leukemia (CLL).

### BRIEF DESCRIPTION OF THE FIGURES

FIG. 1A-C demonstrates that CD44 expression levels on chronic lymphocytic leukemia B cells correlates with features of disease aggressiveness.

FIG. 2A-F demonstrates that anti-CD44 mAb directly induces apoptosis of CLL cells in vitro, with an increased potency against ZapAP-70+ CLL cells.

FIG. 3A-C demonstrates that anti-CD44 mAb-mediated apoptosis in CLL cells is caspase-dependent.

FIG. 4 demonstrates anti-CD44 mAb preferentially induces apoptosis of ZAP-70+ CLL cells, even in the presence of MSC.

FIG. 5A-D demonstrates that anti-CD44 monoclonal antibody blocks HA induced AKT phosphorylation and survival in CLL cells.

FIG. 6A-H demonstrates that anti-CD44 mAb down-modulates CD44 and ZAP-70 protein expression in CLL cells, disrupts the CD44-ZAP-70 complex and abrogates BCR-derived survival signaling.

FIG. 7A-B demonstrates that anti-CD44 mAb impairs CLL cell survival in vivo.

FIG. 8 demonstrates that anti-CD44 mAb can mediate CLL cell phagocytosis but not complement-induced cell death.

FIG. 9 illustrates the effect of anti-CD44 mAb on patients with or without ZAP-70 expression having similar levels of CD44 expression (MFIR) in CLL cells.

FIG. 10A-B depicts the viability of CLL cells treated with anti-CD44 mAb or Rituximab.

### DETAILED DESCRIPTION OF THE INVENTION

The present invention is based on the seminal generation of an anti-CD44 antibody. Additionally, the invention is based on methods of treatment or prevention for hematological malignancies using an anti-CD44 antibody. Further, this invention provides methods of making a CD44 antibody.

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs.

As stated previously, hematological malignancies are cancers that affect blood, bone marrow, and lymph nodes. These malignancies derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

Historically, hematological malignancies have been most commonly divided by whether the malignancy is mainly located in the blood (leukemia) or in lymph nodes (lymphomas). Leukemias include acute lymphoblastic leukemia, acute mylelogenous leukemia, chronic lymphocytic leukemia, chronic myelogenous leukemia and acute monocytic leukemia. Lymphomas include Hodgkin's lymphomas and non-hodgkin's lymphoma.

Acute lymphoblastic leukemia (ALL) is a form of leukemia, or cancer of the white blood cells characterized by excess lymphoblasts. Acute myeloid leukemia (AML), also known as acute myelogenous leukemia, is a cancer of the myeloid line of blood cells, characterized by the rapid growth of abnormal white blood cells that accumulate in the bone marrow and interfere with the production of normal blood cells. Chronic myelogenous (or myeloid) leukemia (CML), also known as chronic granulocytic leukemia (CGL), is a cancer of the white blood cells. It is a form of leukemia characterized by the increased and unregulated growth of predominantly myeloid cells in the bone marrow and the accumulation of these cells in the blood. B-cell chronic lymphocytic leukemia (B-CLL), also known as chronic lymphoid leukemia (CLL), is the most common type of leukemia. CLL affects B cell lymphocytes. Acute monocytic leukemia (AMoL, or AML-M5) is considered a type of acute myeloid leukemia. Hodgkin's lymphoma, previously known as Hodgkin's disease, is a type of lymphoma, which is a cancer originating from white blood cells called lymphocytes. The non-Hodgkin lymphomas (NHLs) are a diverse group of blood cancers that include any kind of lymphoma except Hodgkin's lymphomas.

As stated previously, CLL is generally not curable but is usually treatable with standard chemotherapy regimens that prolong survival. However, there is a population of CLL patients who are refractory or become refractory to standard chemotherapy regimen. The standard of care for CLL includes chemotherapy, biotherapy, radiation therapy, stem cell transplantation, and combinations thereof.

The terms “anti-CD44 antibody”, “anti-CD44”, “CD44 antibody” or “an antibody that binds to CD44” refers to an antibody that is capable of binding CD44 with sufficient affinity such that the antibody is useful as a diagnostic and/or therapeutic agent in targeting CD44. In one embodiment, the anti-CD44 antibody specifically binds CD44.

In one embodiment, the anti-CD44 antibody is a monoclonal humanized antibody. In another embodiment, the humanized antibody specifically binds a constant region of CD44. In a preferred embodiment, the humanized antibody is the RG7356 antibody described in Weigand et al. Cancer Res. 2012 Sep. 1; 72(17):4329-39, which is incorporated herein by reference in its entirety. In another embodiment, the humanized antibody is characterized by internalization into a cell upon binding to CD44 expressed on the surface of the cell.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being known to those of ordinary skill in the art.

A “naked antibody” refers to an antibody that is not conjugated to a heterologous moiety (e.g., a cytotoxic moiety) or radiolabel. The naked antibody may be present in a pharmaceutical formulation.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG.sub.1, IgG₂, IgG₃, IgG₄, IgA₁, and IgA₂. The heavy chain constant domains that correspond to the different classes of immunoglobulins are called α, δ, ε, γ, and μ, respectively.

The term “diabodies” refers to antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6.sup.th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VI, or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “chimeric” antibody refers to an antibody in which a portion of the heavy and/or light chain is derived from a particular source or species, while the remainder of the heavy and/or light chain is derived from a different source or species.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions.

Framework” or “FR” refers to variable domain residues other than hypervariable region (HVR) residues. The FR of a variable domain generally consists of four FR domains: FR1, FR2, FR3, and FR4. Accordingly, the HVRand FR sequences generally appear in the following sequence in VH (or VL): FR1-H1(L1)-FR2-H2(L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “refractory tumor” or “refractory cancer” or “refractory hematological malignancy” is used herein to refer to tumors, cancers, or “hematological malignancies that fail or are resistant to a certain treatment, such as “standard of care” treatment, e.g., treatment with chemotherapeutic agents alone or in combination, biotherapy alone, radiation therapy, stem cell transplantation, or combinations thereof.

The term “standard of care” is used to refer to a treatment process that an ordinary skilled prudent physician uses to treat a certain disease, such as cancer. The standard of care varies depending on the type and stage of cancer, the patient's condition and treatment history, and the like, and will be apparent to those skilled in the art.

In one aspect, the “standard of care” for a hematological malignancy (e.g., chronic lymphocytic leukemia (CLL)) comprises treatment with chemotherapy and/or biotherapy. In one embodiment, the chemotherapy comprises a single chemotherapeutic agent or more than one chemotherapeutic agent. In another embodiment, the chemotherapy comprises a purine nucleoside analog and/or an alkylating agent. In one other embodiment, the biotherapy comprises a monoclonal antibody. In an additional embodiment, the monoclonal antibody is an anti-CD20 antibody. In one embodiment, the anti-CD20 antibody is rituximab.

An “effective amount” of an agent, e.g., a pharmaceutical formulation, refers to an amount effective, at dosages and for periods of time necessary, to achieve the desired therapeutic or prophylactic result.

As described herein, the standard of care for a hematological malignancy (e.g., CLL) includes chemotherapy, biotherapy, radiation therapy, stem cell transplantation, and combinations thereof. Current treatment for CLL includes several different types of chemotherapies given alone or in combination. Typical chemotherapy agents include nucleoside analogs (e.g., purine nucleoside analogs), alkylating agents and monoclonal antibodies. Fludarabine is an example of a purine analog and is frequently used in combination for CLL therapy. Cyclophosphamide and bendamustine are examples of alkylating agents. Biotherapy includes the administration of polypeptide-based agents, including antibodies. In one embodiment, the antibodies are monoclonal or polyclonal antibodies. In another embodiment, the antibodies are chimeric, humanized or human monoclonal antibodies. Anti-CD20 antibodies, such as rituxamab and ofatumumab, and anti-CD52 antibodies, such as alemtuzumab, are used for chemoimmunotherapy for CLL. Other chemotherapy agents include bendamustine, flavopiridol, lenalidomide, vincristine, doxorubicin and prednisone. Typical combinations include FCR (fludarabine, cyclophosphamide and rituxan) and CHOP (cyclophosphamide, vincristine, doxorubicin and prednisone). Alternative treatments for CLL include radiation and stem cell transplantation.

In one aspect, the subject's hematological malignancy is refractory to chemotherapy comprising a purine nucleoside analog and/or an alkylating agent. In one embodiment, the chemotherapy comprises one or more purine nucleoside analogs selected from the group consisting of fludarabine, pentostatin, azathioprine, azathioprine, mercaptopurine, thioguanine, deoxycoformycin, thiamiprine, hydroxyurea, and cladribine. In another embodiment, the chemotherapy comprises one or more alkylating agents selected from the group consisting of nitrogen mustard analogues (e.g., cyclophosphamide, mechlorethamine, chlorambucil, melphalan, ifosfamide, trofosfamide, bendamustine, and estramustine); alkyl sulfonates (e.g., busulfan, treosulfan, and mannosulfan); ethylene imines (thiotepa, altretamine, triaziquone, and carboquone); nitrosoureas (e.g., carmustine, lomustine, semustine, streptozocin, fotemustine, nimustine, and ranimustine); and triazenes (e.g., dacarbazine and temozolomide). In a preferred embodiment, the hematological malignancy is refractory to chemotherapy comprising a purine nucleoside analog (e.g., fludarabine) and/or cyclophosphamide.

In one other aspect, the subject's hematological malignancy is refractory to chemotherapy comprising a steroid. In another embodiment, the chemotherapy comprises a steroid and an alkylating agent. In one embodiment, the steroid is prednisone. In one other embodiment, the alkylating agent is chlorambucil. In an additional embodiment, the chemotherapy comprises prednisone and chlorambucil.

In one other aspect, the subject's hematological malignancy is refractory to chemotherapy comprising a mitotic inhibitor. In another embodiment, the chemotherapy comprises a mitotic inhibitor, an alkylating agent, and a steroid. In one embodiment, the mitotic inhibitor is vincristine sulfate. In another embodiment the alkylating agent is cyclophosphamide. In an additional embodiment, the steroid is prednisone. In one other embodiment, the chemotherapy is cyclophosphamide, vincristine sulfate, and prednisone (CVP).

In another aspect, the subject's hematological malignancy is refractory to (i) chemotherapy comprising a purine nucleoside analog and/or an alkylating agent; and (ii) biotherapy. In one embodiment, the biotherapy comprises monoclonal antibody therapy. In another embodiment, the monoclonal antibody therapy comprises anti-CD20 antibody therapy or anti-CD52 antibody therapy. In a preferred embodiment, the anti-CD20 antibody is rituximab or ofatumumab. In a preferred embodiment, the anti-CD52 antibody is alemtuzumab. In another preferred embodiment, the hematological malignancy is refractory to (i) chemotherapy comprising a fludarabine; and (ii) monoclonal antibody therapy comprising rituximab or ofatumumab. In one other preferred embodiment, the hematological malignancy is refractory to (i) chemotherapy comprising cyclophosphamide; and (ii) monoclonal antibody therapy comprising rituximab. In yet another preferred embodiment, the hematological malignancy is refractory to (i) chemotherapy comprising bendamustine; and (ii) monoclonal antibody therapy comprising rituximab.

In one embodiment, the subject's hematological malignancy is refractory to therapy comprising FCR (fludarabine, cyclophosphamide and rituximab) and bendamustine. In another embodiment, the subject's hematological malignancy is refractory to therapy comprising alemtuzumab. In additional embodiments, the subject's hematological malignancy is refractory to therapy comprising alemtuzumab, chlorambucil, ofatumumab, bendamustine hydrochloride, cyclophosphamide, or fludarabine phosphate. In another embodiment, the subject's hematological malignancy is refractory to therapy comprising chlorambucil and prednisone. In another embodiment, the subject's hematological malignancy is refractory to therapy comprising cyclophosphamide, vincristine sulfate, and prednisone (CVP). In a preferred embodiment, the hematological malignancy is CLL.

As stated previously, CLL cells highly express the protein CD44 on the cell surface. CD44 is a multi-structural glycoprotein involved in many physiological and pathological functions, including cell-cell and cell-matrix adhesion, support of cell migration, presentation of growth factors, chemokines or enzymes to corresponding cell surface receptors or relevant substrates, as well as transmission of signals from the membrane to the cytoskeleton or nucleus [Naor, D., et al. Adv. Cancer Res. 71, 241-319, (1997); Lesley, J., et al. Adv. Immunol. 54, 271-335, (1993)]. This protein participates in a wide variety of cellular functions including lymphocyte activation, recirculation and homing, hematopoiesis, and tumor metastasis. This glycoprotein is known to bind to multiple ligands (e.g. fibrinogen, fibronectin, alanine, collagen), the principal one being hyaluronic acid (HA). CD44 gene transcription is at least in part activated by beta-catenin and Wnt signaling which is linked to tumour development.

CD44 has several variants which are determined by differential splicing of at least 10 variable exons encoding a segment of the extracellular domain and by cell type specific glycosylation.

CD44 is expressed in many types of malignancies and as such, antibodies against CD44 may be very useful in treating malignancies. Such antibodies would disrupt CD44 matrix interactions and by occupying CD44, induce CD44 signaling which can lead to apoptosis. However, due low level endogenous expression of CD44 on normal cells such an antibody would have to be specific for CD44 expressed on malignant cells to avoid undesirable side effects.

In one aspect, therefore, the present invention provides an antibody or antibody fragment which specifically binds to CD44. In an embodiment, the antibody or antibody fragment binds specifically to CD44 expressed on CLL cells.

In various embodiments, the antibody or antibody fragment of the present invention may be a Fab fragment, a F(ab)₂ fragment, an FV fragment, a single chain FV (scFV) fragment, a dsFV fragment, a CH fragment or a dimeric scFV

As used herein, “specific binding” refers to antibody binding to a predetermined antigen. Typically, the antibody binds with an affinity corresponding to a K_{D} of about 10⁻⁸ M or less, and binds to the predetermined antigen with an affinity (as expressed by K_{D}) that is at least 10 fold less, and preferably at least 100 fold less than its affinity for binding to a non-specific antigen (e.g., BSA, casein) other than the predetermined antigen or a closely-related antigen. Alternatively, the antibody can bind with an affinity corresponding to a K_{A} of about 10⁶ M⁻¹, or about 10⁷ M⁻¹, or about 10⁸ M⁻¹, or 10⁹ M⁻¹ or higher, and binds to the predetermined antigen with an affinity (as expressed by K_{A}) that is at least 10 fold higher, and preferably at least 100 fold higher than its affinity for binding to a non-specific antigen (e.g., BSA, casein) other than the predetermined antigen or a closely-related antigen.

The term “kₐ” (M⁻¹ sec⁻¹), as used herein, is intended to refer to the association rate constant of a particular antibody-antigen interaction. The term “K_{A}” (M), as used herein, is intended to refer to the association equilibrium constant of a particular antibody-antigen interaction.

Naturally occurring antibodies are generally tetramers containing two light chains and two heavy chains. Experimentally, antibodies can be cleaved with the proteolytic enzyme papain, which causes each of the heavy chains to break, producing three separate subunits. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding, but is important in later processes involved in ridding the body of the antigen.

Because Fab and F(ab′)₂ fragments are smaller than intact antibody molecules, more antigen-binding domains are available than when whole antibody molecules are used. Proteolytic cleavage of a typical IgG molecule with papain is known to produce two separate antigen binding fragments called Fab fragments which contain an intact light chain linked to an amino terminal portion of the contiguous heavy chain via by disulfide linkage. The remaining portion of the papain-digested immunoglobin molecule is known as the Fc fragment and consists of the carboxy terminal portions of the antibody left intact and linked together via disulfide bonds. If an antibody is digested with pepsin, a fragment known as an F(ab′)₂ fragment is produced which lacks the Fc region but contains both antigen-binding domains held together by disulfide bonds between contiguous light and heavy chains (as Fab fragments) and also disulfide linkages between the remaining portions of the contiguous heavy chains (Handbook of Experimental Immunology. Vol 1: Immunochemistry, Weir, D. M., Editor, Blackwell Scientific Publications, Oxford (1986)).

As readily recognized by those of skill in the art, altered antibodies (e.g., chimeric, humanized, CDR-grafted, bifunctional, antibody polypeptide dimers (i.e., an association of two polypeptide chain components of an antibody, e.g., one arm of an antibody including a heavy chain and a light chain, or an Fab fragment including VL, VH, CL and CH antibody domains, or an Fv fragment comprising a VL domain and a VH domain), single chain antibodies (e.g., an scFv (i.e., single chain Fv) fragment including a VL domain linked to a VH domain by a linker, and the like) can also be produced by methods well known in the art.

To produce an scFv, standard reverse transcriptase protocols are used to first produce cDNA from mRNA isolated from a hybridoma that produces an mAb for CD44 antigen. The cDNA molecules encoding the variable regions of the heavy and light chains of the mAb can then be amplified by standard polymerase chain reaction (PCR) methodology using a set of primers for mouse immunoglobulin heavy and light variable regions (Clackson (1991) Nature, 352, 624-628). The amplified cDNAs encoding mAb heavy and light chain variable regions are then linked together with a linker oligonucleotide in order to generate a recombinant scFv DNA molecule. The scFv DNA is ligated into a filamentous phage plasmid designed to fuse the amplified cDNA sequences into the 5′ region of the phage gene encoding the minor coat protein called g3p. Escherichia coli bacterial cells are than transformed with the recombinant phage plasmids, and filamentous phage grown and harvested. The desired recombinant phages display antigen-binding domains fused to the amino terminal region of the minor coat protein. Such “display phages” can then be passed over immobilized antigen, for example, using the method known as “panning”, see Parmley and Smith (1989) Adv. Exp. Med. Biol. 251, 215-218; Cwirla et al. (1990) Proc. Natl. Acad. Sci. USA 87, 6378-6382, to adsorb those phage particles containing scFv antibody proteins that are capable of binding antigen. The antigen-binding phage particles can then be amplified by standard phage infection methods, and the amplified recombinant phage population again selected for antigen-binding ability. Such successive rounds of selection for antigen-binding ability, followed by amplification, select for enhanced antigen-binding ability in the scFvs displayed on recombinant phages. Selection for increased antigen-binding ability may be made by adjusting the conditions under which binding takes place to require a tighter binding activity. Another method to select for enhanced antigen-binding activity is to alter nucleotide sequences within the cDNA encoding the binding domain of the scFv and subject recombinant phage populations to successive rounds of selection for antigen-binding activity and amplification (see Lowman et al. (1991) Biochemistry 30, 10832-10838; and Cwirla et al. (1990) Proc. Natl. Acad. Sci. USA 87, 6378-6382).

Once an scFv is selected, it can be produced in a free form using an appropriate vector in conjunction with E. coli strain HB2151. These bacteria actually secrete scFv in a soluble form, free of phage components (Hoogenboom et al. (1991) Nucl. Acids Res. 19, 4133-4137). The purification of soluble scFv from the HB2151 bacteria culture medium can be accomplished by affinity chromatography using antigen molecules immobilized on a solid support such as AFFIGEL™ (BioRad, Hercules, Calif.).

Other developments in the recombinant antibody technology demonstrate possibilities for further improvements such as increased avidity of binding by polymerization of scFvs into dimers and tetramers (see Holliger et al. (1993) Proc. Natl. Acad. Sci. USA 90, 6444-6448).

Furthermore, recombinant antibody technology offers a more stable genetic source of antibodies, as compared with hybridomas. Recombinant antibodies can also be produced more quickly and economically using standard bacterial phage production methods.

To produce the antibodies or antibody fragments described herein recombinantly, nucleic acids encoding an antibody or antibody fragments is inserted into expression vectors. The light and heavy chains can be cloned in the same or different expression vectors. The teachings of U.S. Pat. No. 6,287,569 to Kipps et al., incorporated herein by reference in its entirety, and the methods provided herein can readily be adapted by those of skill in the art to create the scFvs of the present invention.

Expression vectors are typically replicable in the host organisms either as episomes or as an integral part of the host chromosome. E. coli is one prokaryotic host particularly useful for expressing antibodies of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired. Mammalian tissue cell culture can also be used to express and produce the antibodies of the present invention (see, e.g., Winnacker, From Genes to Clones VCH Publishers, N.Y., 1987). Eukaryotic cells are preferred, because a number of suitable host cell lines capable of secreting intact antibodies have been developed. Preferred suitable host cells for expressing nucleic acids encoding the immunoglobulins of the invention include: monkey kidney CV1 line transformed by SV40 (COS-7, ATCC CRL 1651); human embryonic kidney line; baby hamster kidney cells (BHK, ATCC CCL 10); Chinese hamster ovary-cells (CHO); mouse sertoli cells; monkey kidney cells (CV1 ATCC CCL 70); african green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and TRI cells.

The vectors containing the polynucleotide sequences of interest can be transferred into the host cell. Calcium chloride transfection is commonly utilized for prokaryotic cells, whereas calcium phosphate treatment or electroporation can be used for other cellular hosts (see, e.g., Sambrook et al., Molecular Cloning: A Laboratory Manual, Cold Spring Harbor Press, 2nd ed., 1989). After introduction of recombinant DNA, cell lines expressing immunoglobulin products are cell selected. Cell lines capable of stable expression are preferred (i.e., undiminished levels of expression after fifty passages of the cell line).

Once expressed, the antibody or antibody fragment of the present invention can be purified according to standard procedures of the art, including ammonium sulfate precipitation, affinity columns, column chromatography, gel electrophoresis and the like (see, e.g., Scopes, Protein Purification, Springer-Verlag, N.Y., 1982). Substantially pure immunoglobulins of at least about 90 to 95% homogeneity are preferred, and 98 to 99% or more homogeneity most preferred.

A labeled antibody or a detectably labeled antibody is generally an antibody (or antibody fragment which retains binding specificity), having an attached detectable label. The detectable label is normally attached by chemical conjugation, but where the label is a polypeptide, it could alternatively be attached by genetic engineering techniques. Methods for production of detectably labeled proteins are well known in the art. Detectable labels known in the art include radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies are well known in the art (see, for example, Harlow and Lane, eds., 1988, Antibodies: A Laboratory Manual, Cold Spring Harbor Laboratory Press, Cold Spring Harbor, N.Y.) Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, pyridoxal, and fluorescein, which can react with specific antihapten antibodies.

Antibodies may be humanized by replacing sequences of the Fv variable region which are not directly involved in antigen binding with equivalent sequences from human Fv variable regions. General reviews of humanized chimeric antibodies are provided by Morrison et al., (Science 229:1202-1207 (1985)) and by Oi et al. (BioTechniques 4:214 (1986)). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from for example, an antibody producing hybridoma. The recombinant DNA encoding the humanized or chimeric antibody, or fragment thereof, can then be cloned into an appropriate expression vector.

Humanized antibodies can alternatively be produced by CDR substitution (U.S. Pat. No. 5,225,539; Jones, Nature 321:552-525 (1986); Verhoeyan et al., Science 239:1534 (1988); and Beidler, J. Immunol. 141:4053-4060 (1988)). Thus, in certain embodiments, the antibody used in the conjugate is a humanized or CDR-grafted form of an antibody produced by the hybridoma having ATCC accession number PTA 2439. In other embodiments the antibody is a humanized or CDR-grafted form of antibody mAb 3E10. For example, the CDR regions can include amino acid substitutions such as 1, 2, 3, 4, 5, 6, 7, 8, 9, or 10 amino acid differences from those shown in the figures. In some instances, there are anywhere from 1-5 amino acid differences.

Such variants include those wherein one or more substitutions are introduced into the heavy chain nucleotide sequence and/or the light chain nucleotide sequence of the antibody or antibody fragment.

The antibodies of the present invention may be conjugated to another molecule. A variety of linkers may be used to link portions of the conjugates described herein. The term “degradable linker” as used herein, refers to linker moieties that are capable of cleavage under various conditions. Conditions suitable for cleavage can include but are not limited to pH, UV irradiation, enzymatic activity, temperature, hydrolysis, elimination, and substitution reactions, and thermodynamic properties of the linkage. The term “photolabile linker” as used herein, refers to linker moieties as are known in the art that are selectively cleaved under particular UV wavelengths. Compounds of the invention containing photolabile linkers can be used to deliver compounds to a target cell or tissue of interest, and can be subsequently released in the presence of a UV source.

The term “linker” as used herein is any bond, small molecule, or other vehicle which allows the substrate and the active molecule to be targeted to the same area, tissue, or cell, for example by physically linking the individual portions of the conjugate.

In certain embodiments, a cleavable or degradable linker may be used. In one embodiment the linker is a chemical bond between one or more substrates and one or more therapeutic moieties. Thus, the bond may be covalent or ionic. An example of a therapeutic complex where the linker is a chemical bond would be a fusion protein. In one embodiment, the chemical bond is acid sensitive and the pH sensitive bond is cleaved upon going from the blood stream (pH 7.5) to the transcytotic vesicle or the interior of the cell (pH about 6.0). Alternatively, the bond may not be acid sensitive, but may be cleavable by a specific enzyme or chemical which is subsequently added or naturally found in the microenvironment of the targeted site. Alternatively, the bond may be a bond that is cleaved under reducing conditions, for example a disulfide bond.

Alternatively, the bond may not be cleavable. Any kind of acid cleavable or acid sensitive linker may be used. Examples of acid cleavable bonds include, but are not limited to: a class of organic acids known as cipolycarboxylic alkenes. This class of molecule contains at least three carboxylic acid groups (COOH) attached to a carbon chain that contains at least one double bond. These molecules as well as how they are made and used is disclosed in Shen, et al. U.S. Pat. No. 4,631,190.

In another embodiment the linker is a small molecule such as a peptide linker. In one embodiment the peptide linker is not cleavable. In a further embodiment the peptide linker is cleavable by base, under reducing conditions, or by a specific enzyme. In one embodiment, the enzyme is indigenous. Alternatively, the small peptide may be cleavable by an non-indigenous enzyme which is administered after or in addition to the therapeutic complex. Alternatively, the small peptide may be cleaved under reducing conditions, for example, when the peptide contains a disulfide bond. Alternatively, the small peptide may be pH sensitive.

The peptide linker may also be useful as a peptide tag (e.g., myc or His₆ (SEQ ID NO: 1)) or may be one or more repeats of the known linker sequence GGGGS (SEQ ID NO: 2). The skilled artisan will recognize that the linker sequence may be varied depending on the polypeptide portions to be linked to form the conjugate. Additional peptide linkers and tags are known in the art, such as epitope tags, affinity tags, solubility enhancing tags, and the like. Examples of various additional tags and linkers that may be used with the present invention include, haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO), and ubiquitin (Ub). Further examples include: poly(L-Gly), (Poly L-Glycine linkers); poly(L-Glu), (PolyL-Glutamine linkers); poly (L-Lys), (Poly L-Lysine linkers). In one embodiment, the peptide linker has the formula (amino acid) n, where n is an integer between 2 and 100, preferably wherein the peptide comprises a polymer of one or more amino acids.

The chemical and peptide linkers can be bonded between the antibody and the conjugate by techniques known in the art for conjugate synthesis, i.e. using genetic engineering, or chemically. The conjugate synthesis can be accomplished chemically via the appropriate antibody by classical coupling reactions of proteins to other moieties at appropriate functional groups.

As used herein, the term “nucleic acid” refers to polynucleotides such as deoxyribonucleic acid (DNA), and, where appropriate, ribonucleic acid (RNA). The term should also be understood to include, as appropriate to the context or as applicable to the embodiment being described, both single-stranded polynucleotides (such as antisense) and double-stranded polynucleotides (such as siRNAs).

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” In the present specification, “plasmid” and “vector” are used interchangeably unless otherwise clear from the context. In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

In one embodiment, the present invention provides a method of producing a protein. The method includes transforming a host cell with an expression construct, and culturing the host cell under conditions suitable for producing the conjugate.

Vectors suitable for use in preparation of proteins and/or protein conjugates include those selected from baculovirus, phage, plasmid, phagemid, cosmid, fosmid, bacterial artificial chromosome, viral DNA, Pl-based artificial chromosome, yeast plasmid, and yeast artificial chromosome. For example, the viral DNA vector can be selected from vaccinia, adenovirus, foul pox virus, pseudorabies and a derivative of SV40. Suitable bacterial vectors for use in practice of the invention methods include pQE70™, pQE60™, pQE-9™, pBLUESCRIPT™ SK, pBLUESCRIPT™ KS, pTRC99a™, pKK223-3™, pDR540™, PAC™ and pRIT2T™. Suitable eukaryotic vectors for use in practice of the invention methods include pWLNEO™, pXTI™, pSG5™, pSVK3™, pBPV™, pMSG™, and pSVLSV40™. Suitable eukaryotic vectors for use in practice of the invention methods include pWLNEO™, pXTI™, pSG5™, pSVK3™, pBPV™, pMSG™, and pSVLSV40™.

Those of skill in the art can select a suitable regulatory region to be included in such a vector, for example from lacI, lacZ, T3, T7, apt, lambda PR, PI, trp, CMV immediate early, HSV thymidine kinase, early and late SV40, retroviral LTR, and mouse metallothionein-I regulatory regions.

Host cells in which the vectors containing the polynucleotides encoding the protein conjugates can be expressed include, for example, a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g. COS-7), or Bowes melanoma cells are all suitable host cells for use in practice of the invention methods.

The expression construct that is delivered typically is part of a vector in which a regulatory element such as a promoter is operably linked to the nucleic acid of interest. The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus. The inducer also can be an illumination agent such as light and light's various aspects, which include wavelength, intensity, fluorescence, direction, and duration.

An example of an inducible promoter is the tetracycline (tet)-on promoter system, which can be used to regulate transcription of the nucleic acid. In this system, a mutated Tet repressor (TetR) is fused to the activation domain of herpes simplex VP 16 (transactivator protein) to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements.

Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include puromycin, adenosine deaminase (ADA), aminoglycoside phosphotransferase (neo, G418, APIA), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

Viral vectors can be used to form the conjugates, and include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

Non-viral vectors can also be used in the subject conjugates. To further illustrate, in one embodiment, the mammalian serum protein that is encoded by the vector is selected from the group consisting of a tissue-type plasminogen activator, a receptor of a tissue-type plasminogen activator, a streptokinase, a staphylokinase, a urokinase, and coagulation factors. The invention also provides a method for treating associated with the formation of clots in its circulation, including the step of administering to the mammal a conjugate that causes the recombinant expression and secretion into the blood, such as from transduced muscle cells, of a therapeutically effective amount of such a mammalian serum protein.

In another aspect, the present invention provides a pharmaceutical composition including the antibody or antibody fragment of the invention and optionally a pharmaceutically acceptable carrier.

In another aspect, the present invention provides a method for detecting CD44 protein in a sample. The method comprising contacting the sample with a labeled CD44 antibody or antibody fragment and detecting the immunoreactivity between the detectably labeled antibody and CD44 in the sample.

In another aspect, the present invention provides a method for treating a hematological malignancy in a human subject using an antibody of the invention. The method comprises administering an effective amount of an antibody or antibody fragment of the invention to a subject in need thereof.

In another aspect, the present invention provides a method for treating or preventing CLL in which an antibody binding of CD44 on CLL cells confers a survival advantage thereon by administering a CD44 antibody of the invention to a subject in need thereof.

In another aspect, the present invention provides a method of treating a hematological malignancy in a subject in need thereof, the method comprising administering to the subject an effective amount of an antibody to CD44, wherein the subject's hematological malignancy is refractory to chemotherapy and/or biotherapy. In one embodiment, the subject's hematological malignancy is refractory to therapy, wherein the therapy comprises (i) chemotherapy comprising a purine nucleoside analog and/or an alkylating agent, and/or (ii) biotherapy comprising monoclonal antibody therapy. In another embodiment, the hematological malignancy is leukemia, preferably lymphocytic leukemia. In one other embodiment, the lymphocytic leukemia is B-cell chronic lymphocytic leukemia (CLL).

In another aspect, the present invention provides a method of treating B-cell chronic lymphocytic leukemia (CLL) in a subject in need thereof, the method comprising administering to the subject an effective amount of an antibody to CD44, wherein the subject's CLL is refractory to chemotherapy and/or biotherapy. In one embodiment, the subject's CLL is refractory to therapy, wherein the therapy comprises (i) chemotherapy comprising a purine nucleoside analog and/or an alkylating agent, and/or (ii) biotherapy comprising monoclonal antibody therapy.

In a further aspect, the invention provides for the use of an anti-CD44 antibody in the manufacture or preparation of a medicament. In one embodiment, the medicament is for treatment of a hematological malignancy in a subject, wherein the hematological malignancy is refractory to chemotherapy and/or biotherapy. In another embodiment, the medicament is for use in a method of treating hematological malignancy comprising administering to an subject having hematological malignancy an effective amount of the medicament, wherein the hematological malignancy is refractory to chemotherapy and/or biotherapy. In certain embodiments, the hematological malignancy is a leukemia. In other embodiments, the leukemia is a lymphocytic leukemia. In an additional embodiment, the lymphocytic leukemia is B-cell chronic lymphocytic leukemia (CLL).

In one other embodiment, the present invention provides an antibody that binds to CD44 for use in a method of treating a hematological malignancy, wherein the antibody is internalized by and inhibits the growth of a CD44-expressing cell that is associated with the hematological malignancy. In another embodiment, the present invention provides the use of an antibody that binds CD44 for the manufacture of a medicament for treating a hematological malignancy, wherein the antibody is internalized by and inhibits the growth of a CD44-expressing cell that is associated with the hematological malignancy. In an additional embodiment, the antibody is conjugated to another molecule, e.g., a therapeutic molecule. In some embodiment, the antibody is conjugated to another molecule via a linker. In some embodiments, the hematological malignancy is CLL. In another embodiment the CD44-expressing cell is a B cell. In a preferred embodiment, the B cell is a CLL cell. In another embodiment, the anti-CD44 antibody specifically binds to CD44.

In various embodiments, the method of preventing or treating a hematological malignancy may further comprise an additional therapeutic agent. Such therapeutic agents may include purine analogs, alkylating agents, monoclonal antibodies as well as other chemotherapies. Examples of purine analogs include fludarabine, pentostatin, azathioprine, azathioprine, mercaptopurine, thioguanine, and cladribine. Examples of alkylating agents include cyclophosphamide, mechlorethamine or mustine, uramustine or uracil, melphalan, chlorambucil, ifosfamide, nitrosoureas, carmustine, lomustine, streptozocin and busulfan.

Examples of monoclonal antibodies includes, but are not limited to, Anti-EGFr antibodies (e.g., panitumamab, Erbitux (cetuximab), matuzumab, IMC-IIF 8, TheraCIM hR3), denosumab, Avastin (bevacizumab), Anti-HGF antibodies, Humira (adalimumab), Anti-Ang-2 antibodies, Herceptin (trastuzumab), Remicade (infliximab), Anti-CD20 antibodies, rituximab, Synagis (palivizumab), Mylotarg (gemtuzumab oxogamicin), Raptiva (efalizumab), Tysabri (natalizumab), Zenapax (dacliximab), NeutroSpec (Technetium (.sup.99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab, ofatumumab and GA 101.

Examples of other chemptherapuetic agents includes, but is not limited to, busulfan, improsulfan, piposulfan; benzodopa, carboquone, meturedopa, uredopa; ethylenimines, altretamine, triethylenemelamine, trietylenephosphoramide, triethiylenethiophosphoramide, trimethylolomelamine; bullatacin, bullatacinone delta-9-tetrahydrocannabinol, beta-lapachone; lapachol; colchicines; betulinic acid; topotecan, CPT; bryostatin; callystatin; CC-1065 (including its adozelesin, carzelesin and bizelesin synthetic analogues); podophyllotoxin; podophyllinic acid; teniposide; cryptophycins; dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, adriamycin, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; taxoids. doxetaxel; chloranbucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; capecitabine, oxalipltatin, bendamustine, flavopiridol, lenalidomide, cisplatin, cytarabine, mitoxantrone, dexamthasone.

In another aspect of the invention combinations of two or more of the above such as CHOP, an abbreviation for a combined therapy of cyclophosphamide, doxorubicin, vincristine, and prednisolone; FC, an abbreviation for fludarabine and cyclophosphamide, FRC, an abbreviation for fludarabine, cyclophosphamide and rituxan, and FOLFOX, an abbreviation for a treatment regimen with oxaliplatin combined with 5-FU and leucovovin may be used to treat or prevent a hematological malignancy.

In another aspect, the present invention provides a method of targeting an antibody to a cell having an CD44 receptor. The method includes contacting the cell with an antibody of the invention

In another aspect, the present invention provides a method of monitoring a therapeutic regimen or disease progression for treating a subject having or at risk of having an hematological malignancy using an antibody of the invention. A further embodiment involves determining whether an increase or decrease in an amount of CD44 protein has occurred in comparison to a control level of CD44, the increase or decrease as a result of administration of an antibody of the invention to a subject or disease progression.

In another aspect, the present invention provides a kit to detect the presence of CD44 protein in a sample from a subject that is known or suspected to contain hematological malignant cells. The kit includes an antibody of the invention and instructions for its use in an assay environment.

As discussed herein, the antibodies or antibody fragments of the invention may include humanized antibodies, and can be combined for therapeutic use with additional active or inert ingredients, e.g., in conventional pharmaceutically acceptable carriers or diluents, e.g., immunogenic adjuvants, and optionally with adjunctive or combinatorially active molecules such as anti-inflammatory and anti-fibrinolytic drugs.

In other embodiments, the antibodies or antibody fragments described herein are coordinately administered with, co-formulated with, or coupled to (e.g., covalently bonded) a combinatorial therapeutic agent, for example a radionuclide, a differentiation inducer, a drug, or a toxin. Various known radionuclides can be employed, that are well known in the art. Useful drugs for use in such combinatorial treatment formulations and methods include methotrexate, and pyrimidine and purine analogs. Suitable differentiation inducers include phorbol esters and butyric acid. Suitable toxins include ricin, abrin, diptheria toxin, cholera toxin, gelonin, Pseudomonas exotoxin, Shigella toxin, and pokeweed antiviral protein.

In carrying out various assay, diagnostic, and therapeutic methods of the invention, it is desirable to prepare in advance kits comprises a combination of the antibodies or antibody fragments as described herein with other materials. For example, in the case of sandwich enzyme immunoassays, kits of the invention may contain an antibody that specifically binds CD44 optionally linked to an appropriate carrier, a freeze-dried preparation or a solution of an enzyme-labeled monoclonal antibody which can bind to the same antigen together with the monoclonal antibody or of a polyclonal antibody labeled with the enzyme in the same manner, a standard solution of purified CD44, a buffer solution, a washing solution, pipettes, a reaction container and the like. In addition, the kits optionally include labeling and/or instructional materials providing directions (i.e., protocols) for the practice of the methods described herein in an assay environment. While the instructional materials typically comprise written or printed materials, they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials.

### Recombinant Methods

Anti-CD44 antibodies described herein may be produced using recombinant methods and compositions, e.g., as described in U.S. Pat. No. 4,816,567. In one embodiment, isolated nucleic acid encoding an anti-CD44 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20 cell). In one embodiment, a method of making an anti-CD44 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD44 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Assays

Anti-CD44 antibodies provided herein may be identified, screened for, or characterized for their physical/chemical properties and/or biological activities by various assays known in the art. The identification, screening, and characterization may be via binding assays and other assays.

In one aspect, an anti-CD44 antibody of the invention is tested for its CD44 binding activity, e.g., by known methods such as ELISA, Western blot, etc. Numerous types of competitive binding assays can be used to determine if an anti-CD44 antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In certain embodiments, binding is inhibited by at least 80-85%, 85-90%, 90-95%, 95-97%, or 97% or more.

In one aspect of the invention, competition assays may be used to identify an antibody that competes with the RG7356 anti-CD44 antibody described herein (e.g., in Example 4) for binding to CD44. In certain embodiments, such a competing antibody binds to the same epitope (e.g., a linear or a conformational epitope) that is bound by the RG7356 anti-CD44. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized CD44 is incubated in a solution comprising a first labeled antibody that binds to CD44 (e.g., the RG7356 anti-CD44 antibody) and a second unlabeled antibody that is being tested for its ability to compete with the first antibody for binding to CD44. The second antibody may be present in a hybridoma supernatant. As a control, immobilized CD44 is incubated in a solution comprising the first labeled antibody but not the second unlabeled antibody. After incubation under conditions permissive for binding of the first antibody to CD44, excess unbound antibody is removed, and the amount of label associated with immobilized CD44 is measured. If the amount of label associated with immobilized CD44 is substantially reduced in the test sample relative to the control sample, then that indicates that the second antibody is competing with the first antibody for binding to CD44. See Harlow and Lane (1988) Antibodies: A Laboratory Manual ch. 14 (Cold Spring Harbor Laboratory, Cold Spring Harbor, N.Y.).

In another aspect, suitable anti-CD44 antibodies are identified, screened, and characterized for the ability to inhibit the survival of cells associated with a hematological malignancy. In one embodiment, the hematological malignancy is leukemia, preferably lymphocytic leukemia. In a preferred embodiment, the lymphocytic leukemia is CLL. The ability of an anti-CD44 antibody to inhibit the survival of cells associated with a hematological malignancy, e.g., CLL cells, can be verified using the protocols and assays described herein, e.g., Example 4.

### Articles of Manufacture

In another aspect of the invention, an article of manufacture containing materials useful for the treatment, prevention and/or diagnosis of the hematological malignancies described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the hematological malignancy and may have a sterile access port (for example the container may be an intravenous solution bag or a vial having a stopper pierceable by a hypodermic injection needle). At least one active agent in the composition is an anti-CD44 antibody of the invention. The label or package insert indicates that the composition is used for treating the condition of choice. Moreover, the article of manufacture may comprise (a) a first container with a composition contained therein, wherein the composition comprises an anti-CD44 antibody of the invention; and (b) a second container with a composition contained therein, wherein the composition comprises an additional therapeutic agent. In certain embodiments, the second container comprises a second therapeutic agent, such as one of the additional therapeutic agents described herein.

The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

All references cited herein, including patent applications and publications, are incorporated by reference in their entirety.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### Example 1

This example illustrates the toxic effect of anti-CD44 antibody are specific for CLL cells.

Lymphocytes were collected from 32 CLL patients and 4 healthy volunteers. These cells were cultured under standard conditions. Sub-microgram amounts of a CD44 antibody were administered to the cells. The antibody was directly cytotoxic for lymphocytes from the CLL patients but had no effect on the lymphocytes from the healthy volunteers.

### Example 2

This example illustrates the toxic effect of anti-CD44 antibody on CLL cells is not affected by co-culturing with mesenchymal cells.

Lymphocytes were collected from 32 CLL patients and 4 healthy volunteers. These cells were cultured under standard conditions with mesenchymal cells. Sub-microgram amounts of a CD44 antibody were administered to the cells. The antibody was directly cytotoxic for lymphocytes from the CLL patients but had no effect on the lymphocytes from the healthy volunteers. Normally mesenchymal cells can support CLL-cell survival in vitro which didn't occur in the presence of the CD44 antibody.

### Example 3

This example illustrates the clearance of CLL cells grafted into immune deficient mice.

CLL cells were grafted into immune deficient RAG-2−/−/yc−/− mice. The mice were then an administered a CD44 antibody. The results indicated that as little as 1 mg/kg of this mAb resulted in the complete clearance of engrafted CLL cells, an affect not observed in control treated animals.

### Example 4

In this study, the expression level of surface CD44 was evaluated and the efficacy of a newly developed humanized anti-CD44 monoclonal antibody (RG7356, Roche) was tested for the ability to inhibit CLL cell survival in vitro and in vivo. The mechanism of anti-CD44 antibody action also was explored.

### Materials and Methods

Human Samples.

Samples were collected by the CLL Research Consortium (CRC) after informed consent and obtained from patients fulfilling diagnostic criteria for CLL. These samples had more than 95% CD19+/CD5+ cells by flow cytometry. ZAP-70 expression and IgVH gene mutational status were assessed as previously described (Rassenti et al., N Engl J Med 2004, 351:893-901). Buffy coat samples from healthy donors were obtained from the San Diego Blood Bank. Peripheral blood mononuclear cells (PBMCs) were isolated by density centrifugation with Ficoll-Hypaque (Pharmacia, Uppsala, Sweden), resuspended in 90% fetal calf serum (FCS) and 10% dimethylsulfoxide (DMSO) for viable storage in liquid nitrogen.

Reagents.

Humanized anti-CD44 Mab (RG7356) was a gift from Roche (Canada). Control human IgG was purchased from Cell Science. Hyaluronic acid (HA) with high molecular weight (>950 kDa) was purchased from R&D system. Z-VAD-FMK was purchased from BD. RG7356 was conjugated with Alex647 using Alex Fluor® 647 Protein Labeling Kit (Invitrogen) according to manufacturer's instruction, anti-IgM-FITC was purchased from BD, Alex488-conjugated Zap70 was purchased from Caltag Laboratories for flow cytometry analysis.

Western Blot Analysis and Immunoprecipitation.

Primary CLL cells and PBMCs from healthy donors were treated as indicated below. Cells were washed with PBS twice and lysed in lysis buffer (1% NP40, 50 mM Tris-HCl, pH7.5, 100 mM NaCl, 5 mM EDTA) containing protease inhibitors (Roche). The protein concentration was determined using a bicinchoninic acid protein assay (Pierce, Rockford, Ill.). Equal amount of proteins from each sample were resolved by SDS-PAGE followed by immunoblotting with antibodies specific for CD44 (Roche), ZAP-70 (BD Transduction Lab), phospho-AKT (Ser473), AKT, (Cell Signaling Technology), PARP (BD Biosicence), and β-Actin (Santa Cruze Biotechnology). Horseradish peroxidase-conjugated anti-IgG (Cell Signaling Technology) was used as the secondary antibody. The membranes were developed by a chemiluminescence system (Thermo Fisher scientific) and documented by autoradiography.

For immunoprecipitation, cell lysate from each sample was first incubated with protein-A Sepharose beads (50% slurry) for 3 hrs at 4° C., then added the indicated antibodies and further incubated for 1 hr at 4° C. Bound beads were washed four times in lysis buffer before adding SDS sample buffer and subjected to SDS-PAGE and immunoblotting as described above.

Phospho-AKT/Total AKT ELISA.

Levels of p-AKT and total AKT were measured using p-AKT (Ser473) and AKT sandwich ELISA kits (R&D system) according to manufacture instruction. Briefly, cells are fixed, permeabilized in the well of 96-well plates and incubated with two primary antibodies derived from different species. Two secondary antibodies recognizing the different species are labeled with either horseradish-peroxidase (HRP) or alkaline phosphatase (AP), and two spectrally distinct fluorogenic substrate for either HRP or AP were used for detection. The fold change in p-AKT level was calculated based on the fluorescence intensity of p-AKT divided by that of total AKT relative to non-treatment control.

Cell Viability Assay.

Cell viability was determined by the analysis of mitochondrial transmembrane potential (ΔΨm) using 3,3′-dihexyloxacarbocyanine iodine (DiOC6; Invitrogen) and by cell membrane permeability to propidium iodide (PI; Sigma). Primary CLL cells were harvested and transferred to FACS tubes containing 100 μL FACS buffer with 60 nM DiOC6 and 10 μg/mL PI. Cells were incubated at 37° C. for 20 min and analyzed within 30 min by flow cytometry using a FACSCalibur (Becton Dickinson). Fluorescence was recorded at 525 nm (FL-1) for DiOC₆ and at 600 nm (FL-3) for PI. Data was analyzed using the FlowJo 7.2.2 software (Tree Star). The viable cells were determined by calculating the percentage of the PI⁻/DiOC6^{hi} populations.

Co-Culture of CLL Cells with Marrow Stromal Cells (MSCs).

When indicated, CLL cells were co-cultured on a layer of MSCs, which were derived in vitro from the marrow of CLL patients as described. Fecteau et al: Mol Med 2012, 18:19-28). MSCs between passages 2 and 6 were plated at 1000 cells/cm² in 96-well flat-bottom tissue culture plate 2 to 3 days prior to the addition of 1×10⁶ CLL cells per mL (2×10⁵ cells per well) and RG7356 or isotype control antibodies (Sigma) at the indicated concentrations. CLL cell viability was determined using PI and DIOC6 staining.

Detection of CD44 Internalization.

CLL cells were incubated with Alex647-conjugated humanized anti-CD44 antibody on ice for 20 minutes. After washing two times, cells were either left on ice or incubated at 37° C. for indicated time to facilitate internalization. Then mean fluorescence intensity (MFI) was determined via flow cytometry analysis.

Calcium Flux Measurement.

2×10⁶ CLL cells were loaded with 2 uM Fluo-4AM (Molecular Probes) in Hanks balanced salt solution (HBSS) without Ca⁺⁺ and Mg⁺⁺ and incubated for 30 minutes at 37° C. Cells were washed twice with HBSS and then suspended in 1 mL of deficient RPMI. Fluorescence of the cellular suspension was recorded by flow cytometer (Becton Dickinson). Cells were kept at 37° C. for IgM stimulation. Data were analyzed using FlowJo software.

Human CLL Xenograft Animal Study.

Six to eight weeks old RAG2^{-/-}γc^{-/-} mice (obtained from Dr. Catriona Jamieson, University of California San Diego) were housed in laminar-flow cabinets under specific pathogen-free conditions and fed adlibrium. All experiments on mice were conducted in accordance with the guidelines of National Institutes of Health (NIH; Bethesda, Md., USA) for the care and use of laboratory animals. The study protocol was approved by UCSD and Medical Experimental Animal Care Committee (USA). PBMC from primary CLL patients were prepared in AIM-V serum-free medium and 2×10⁷ viable cells were injected to the peritoneal cavity of each mouse. Various doses of the antibody were injected i.p. the next day. Seven days later, peritoneal lavage (PL) was extracted by injecting the cavity with a total volume of 12 mL DPBS. Total recovery of the PL cells was determined using Guava counting. Subsequently, cells were blocked with both mouse and human Fc blocker for 30 mm at 4° C., stained with various human cell surface markers, e.g., CD19, CD5, CD45, and processed for FACS analysis. To calculate the final number of residual CLL cells in PL, the percentage of CLL cells detected by FACS analysis was back calculated to the acquired viable events and then multiplied by the total PL cell counts. Residual CLL cells from human IgG treated mice were set as baseline as 100%. Each treatment group included at least 3 mice and the data were presented as mean±SEM.

Antibody-Dependent Cellular Phagocytosis (ADCP) Assay.

Macrophages were collected from the peritoneal cavity of Rag2^{-/-}γc^{-/-} mice after thioglycolate injection 4 days later and used for the in vitro ADCP assay. Briefly, viably frozen CLL PBMCs were resuspended in RPMI 1640 containing 2% (v/v) FBS Low IgG, 100 U/mL penicillin, and 100 μg/mL streptomycin, and distributed into 96-well Flat-bottom plates (Corning) at a density of 3×10⁴ cells/well along with the indicated concentrations of anti-CD44 antibody, rituximab or isotype control. The plates were incubated on ice for 30 min prior to the addition of the macrophages (1.5×10⁵ cells per well) for an effector to target ratio of 5:1. After 3 h of incubation at 37° C., 5% CO₂, the cells were collected and analyzed by flow cytometry using a FACSCalibur Instrument (BD). Samples containing CLL cells only and macrophages only were used to set up appropriate gating of live CLL cells, based on which the fraction of live CLL cells remaining was determined using FlowJo analytical software.

Complement-Mediated Cytotoxicity Assay (CDC).

ZAP-70-CLL cells were isolated, washed, and resuspended in RPMI 1640 containing 10% (v/v) FBS, 100 U/mL penicillin, and 100 μg/mL streptomycin, and distributed into 96-well U-bottom plates (Corning) at a density of 1×10⁵ cells/well. After incubation for 1 h on ice with 10 μg/mL of antibodies, the cells were harvested, washed once with PBS to remove unbound antibodies, and incubated with 20% complement from 3-4-week-old rabbits for 2 h at 37° C. in 5% CO2. CLL cell viability was determined by flow cytometry following DiOC6 and PI stainings as described above.

Statistical Analysis.

Statistical significance was determined using paired or unpaired Student's t test or one-way ANOVA followed by Dunnett's multiple comparisons test. The correlation between ZAP-70 expression versus viability of cells treated with anti-CD44 antibody was analyzed using Pearson coefficiency. Unless indicated otherwise, data are presented as either the mean SEM or the median SEM.

### Results

CD44 is Highly Expressed in CLL Cells, Particularly in ZAP-70+ Cells.

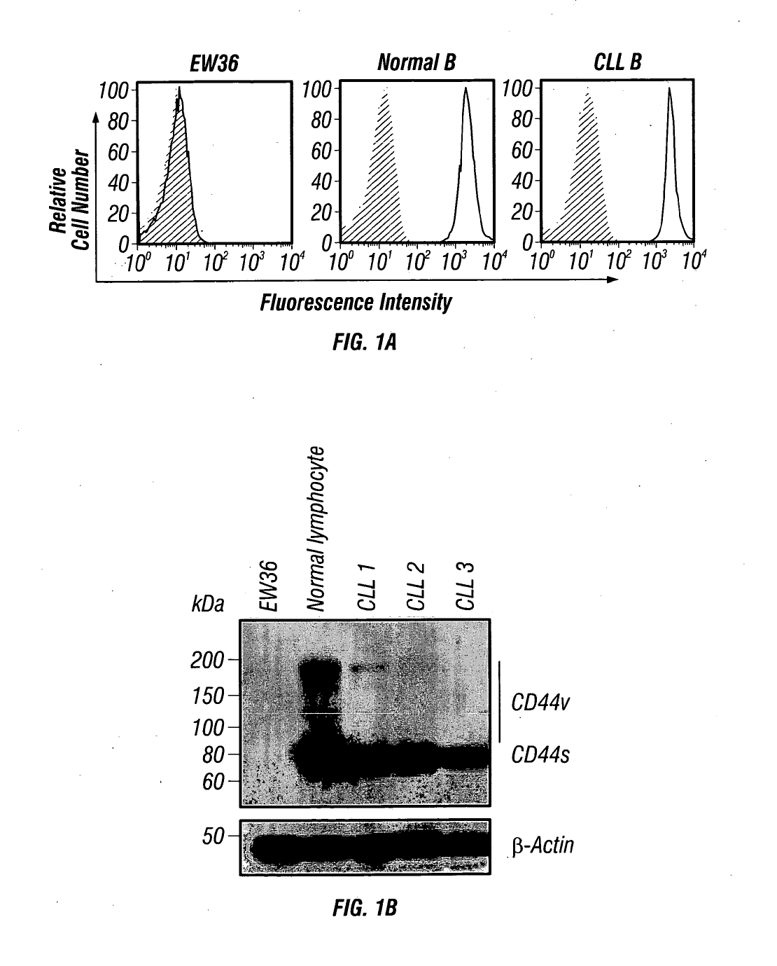

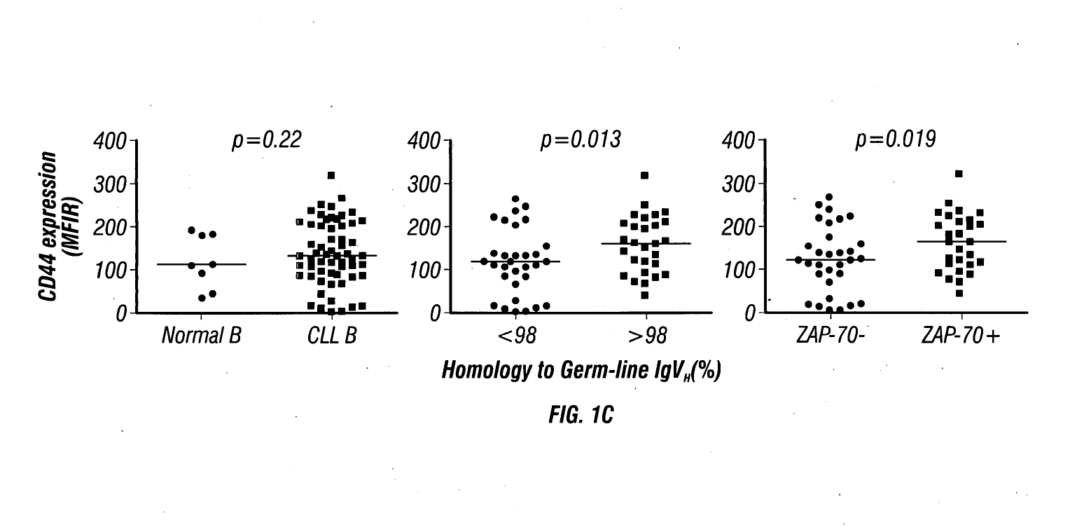

Leukemia B cells from 59 patients with CLL and normal B cells from 8 healthy donors were analyzed for expression of CD44 protein using humanized anti-CD44 mAb (RG7356) either conjugated to Alexa647 for flow cytometry or unconjugated for immunoblot analysis. FIG. 1A depicts fluorescence histograms of human lymphoma B cell-line (EW36), normal B cells and CLL cells stained with humanized CD44 mAb (open histograms) or control mAb (shaded histograms). High levels of CD44 expression were detected in both CD19+CD5+ CLL B cells and CD19+ normal B cells by flow, but not detectable in Lymphoma B cell line EW36 (FIG. 1A). FIG. 1B depicts immunoblot analysis of lysates from EW36, peripheral blood mononuclear cells (PBMC) from healthy adult or CLL patients using humanized CD44 mAb specific for human CD44 or Ab for β-Actin. In Western blot analysis, various CD44 isoforms and standard CD44 protein have all been detected by RG7356 in either normal peripheral blood mononuclear cell (PBMC) or CLL B cells (FIG. 1B). It appeared that standard CD44 is predominant in both CLL cells and normal lymphocyte cells (FIG. 1B). FIG. 1C depicts PBMCs from healthy adult (n=8) or patients with CLL (n=59) stained with humanized anti-CD44 mAb or control mAb as in panel A. MFIR (Mean Fluorescence Intensity Ratio) is obtained by dividing the mean fluorescence intensity (MFI) of CD44 mAb by MFI of control mAb. Each dot represents the expression of CD44 from an individual CLL patient sample gated on CD19+CD5+ B cells or a healthy adult sample gated on CD 19+ B cells (left). CD44 expression levels were correlated with clinical features of CLL according to the extent of somatic mutations in IgV_{H} genes (middle) or to the level of ZAP-70 expression (right) as described (Chiorazzi et al., N Engl J Med 2005, 352:804-815). The line indicates the median CD44 expression level by each group. P<0.05 indicates statistical significance of the differences in the collective CD44 expression between the two groups, as calculated using Student's t test. Although similar levels of CD44 were detected in normal B cells (median of the mean fluorescence intensity ratio (MFIR)=111.7) and CLL B cells (MFIR=131.9) (FIG. 1C, right panel), the levels of CD44 expression in CLL B cells with unmutated IGVH gene were significantly higher than that with mutated IGVH gene (FIG. 1C, middle panel; MFIR median=161.2 vs 118.5, respectively; p=0.013). Furthermore, CLL B cells with ZAP-70 expression also showed significantly higher CD44 expression level compare to those with low or no ZAP-70 expression (FIG. 1C, left panel); MFIR median=161.2 vs 118.7, respectively; p=0.019).

Sensitivity of CLL Cells to RG7356-Induced Direct Apoptosis.

Because CD44 expression level correlates with ZAP-70 expression in CLL cells, patients with high levels of ZAP-70 expression might be more responsive to RG7356 treatment. To test this hypothesis, primary CLL cells from a total of 28 patients (ZAP-70+, n=16; ZAP-70−, n=12) and PBMCs from 6 healthy donors were incubated with RG7356 with increasing concentrations for 24 hours. Induction of apoptosis was analyzed by flow cytometry based on DiOC₆/PI staining.

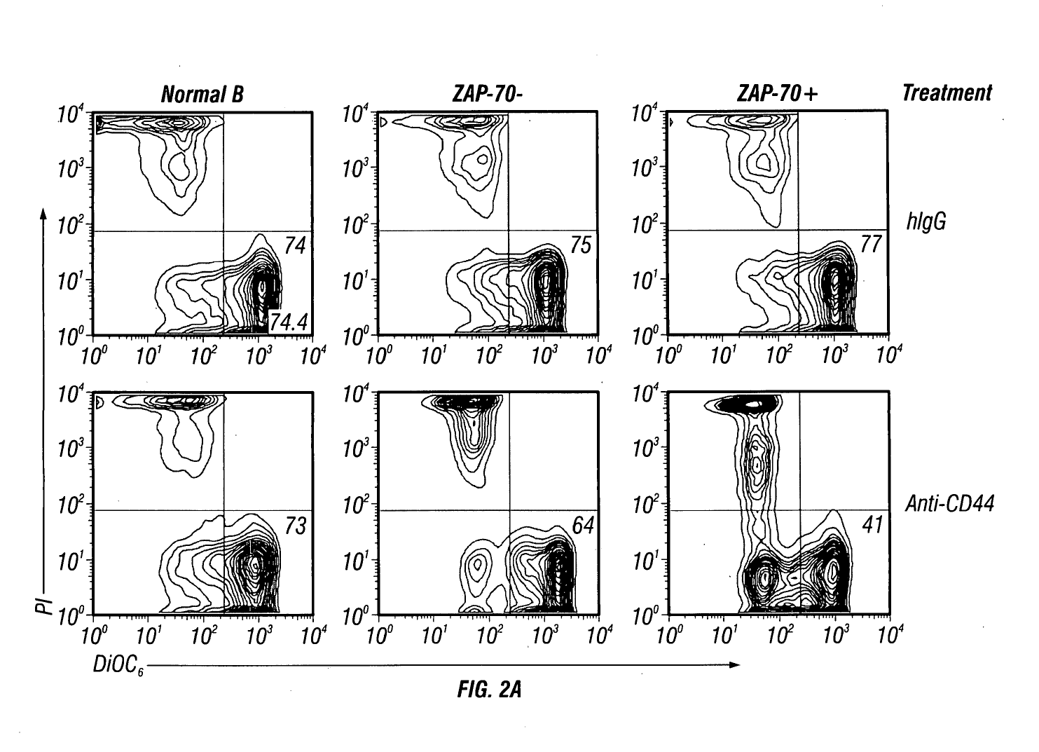

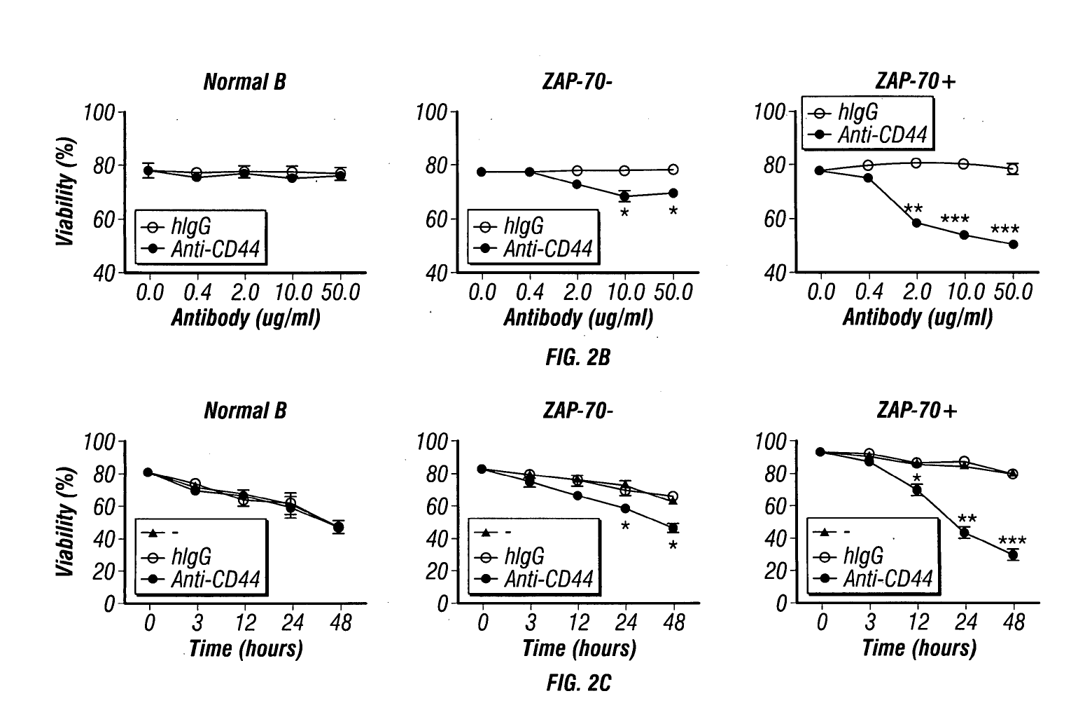

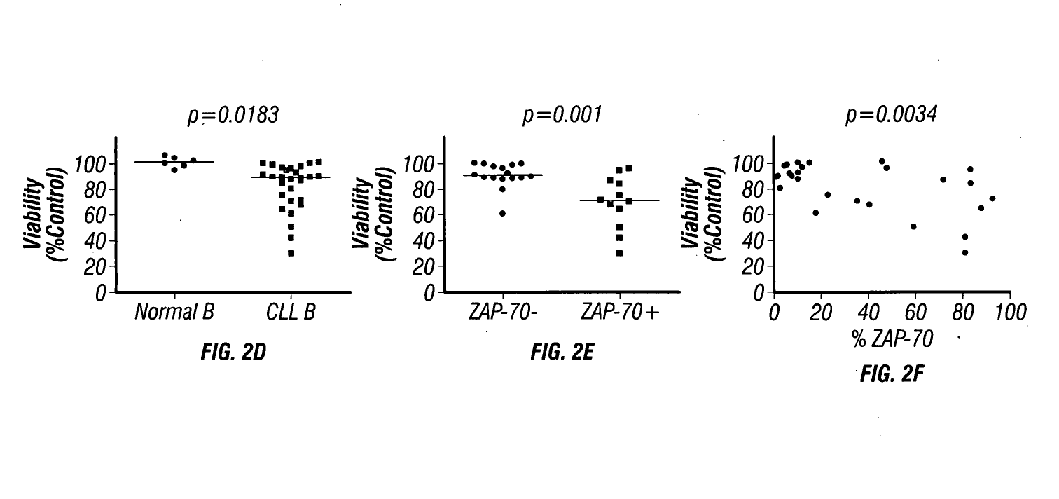

As depicted in FIG. 2, CLL cells or normal PBMC were cultured in the presence of anti-CD44 or hIgG control mAb at the indicated concentrations and time period. The cells were harvested and stained with DiOC₆/PI to measure viability by flow cytometry. Normal PBMC was also stained for CD 19 expression to evaluate cell death in the B cell population. FIG. 2A presents contour maps from 2 representative CLL samples incubated with 50 ug/ml mAb for 24 hours. The relative DiOC6 and PI fluorescence intensities are depicted on the X and Y axis respectively. Cells in the lower right quadrant which are DiOC₆ bright and PI negative are viable and those numbers were used for the generation of plots shown. FIG. 2B depicts CLL cells separated according to ZAP-70 expression or normal PBMC were cultured in the presence of increasing concentrations of mAb and harvested after 24 hours for analysis of viability as above. Data shown are mean+/−SEM of representative samples in triplicate. * indicates P<0.05, ** indicates P<0.01, *** indicates P<0.001 (Student's t test). FIG. 2C shows cells cultured in the presence or absence of (50 ug/ml) mAb and harvested at indicated time for analysis of viability as above. Data shown are mean+/−SEM of representative samples in triplicate. * indicates P<0.05, ** indicates P<0.01, *** indicates P<0.001 (Student's t test). In FIG. 2D each dot represents the relative viability of cells from one patient cultured with 50 ug/ml anti-CD44 mAb for 24 hours. The percent viable cells have been normalized to the viability of control mAb treated cells. The line indicates the median viability of cells treated with anti-CD44 mAb by the group. N=6 for normal and n=28 for CLL cells FIG. 2E depicts the percent viable cells remaining following CD44 mAb exposure depicted in D are presented in function of ZAP-70 status, using the standard 20% expression as a cut off. Viability of ZAP-70+ CLL cells (n=12) treated with anti-CD44 mAb is significantly lower than that of ZAP-70− CLL cells (n=16) treated with anti-CD44 mAb. P=0.001 (Student's t test). FIG. 2F depicts the percent viable cells remaining following anti-CD44 mAb exposure depicted in D are presented in function of the percent of cells expressing ZAP-70 for each sample. ZAP-70 expression level is associated with viability of cells treated with anti-CD44 mAb. Pearson R=−0.5345, P=0.0034, n=28.

As little as 2 μg/ml of RG7356 induced apoptosis in CLL cells, whereas little or no effect was observed in normal B cells treated with RG7356 at concentration as high as 50 μg/ml or with human IgG control antibody (FIG. 2A-B). In addition, induction of apoptosis in ZAP-70+ CLL cells was dose dependent. These results suggest that RG7356 has selective cytotoxicity to CLL cells particularly in those express high levels of ZAP-70.

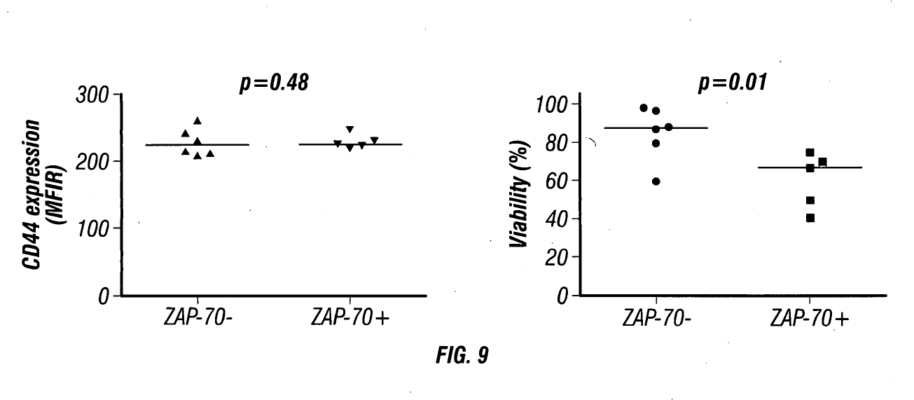

To study the kinetics of the cytotoxicity induced by RG7356, cells were treated with 50 μg/ml of RG7356 and analyzed at various time points. Increased cell death occurred as soon as 3 hours after RG7356 treatment in CLL samples and continued over time as compared to cells without treatment or treated with control hIgG (FIG. 2C). Among different CLL samples, ZAP-70+ cells were significantly more sensitive to RG7356-induced apoptosis than ZAP-70− ones regardless comparable levels of CD44 expression (FIG. 2E and FIG. 9). Furthermore, a significant reverse association between the viability of CLL cells treated with RG7356 at 24 hours and the level of ZAP-70 expressed by each of the individual samples was observed (FIG. 2F, Pearson R=−0.5345, P=0.0034). In contrast, no difference was observed in normal B cells regardless of treatments for up to 48 hours (FIG. 2C-D) suggesting that this specific CD44 Mab exhibits selectivity against CLL cells and is relatively safe to normal B cells.

RG7356-Mediated Apoptosis is Caspase-Dependent.

Consistent with a recent report that cell death mediated by anti-CD44 mAb was marked by the loss of mitochondrial transmembrane potential (Gupta et al. J Cell Mol Med 2009, 13:1004-1033), our data also confirms the induction of apoptosis by RG7356 in CLL cells. To further investigate the mechanism by which RG7356 induces CLL cell death, cells were treated and stained with annexin V and 7AAD for FACS analysis or lysed for SDS-PAGE to access the cleavage of PARP by Western blot.

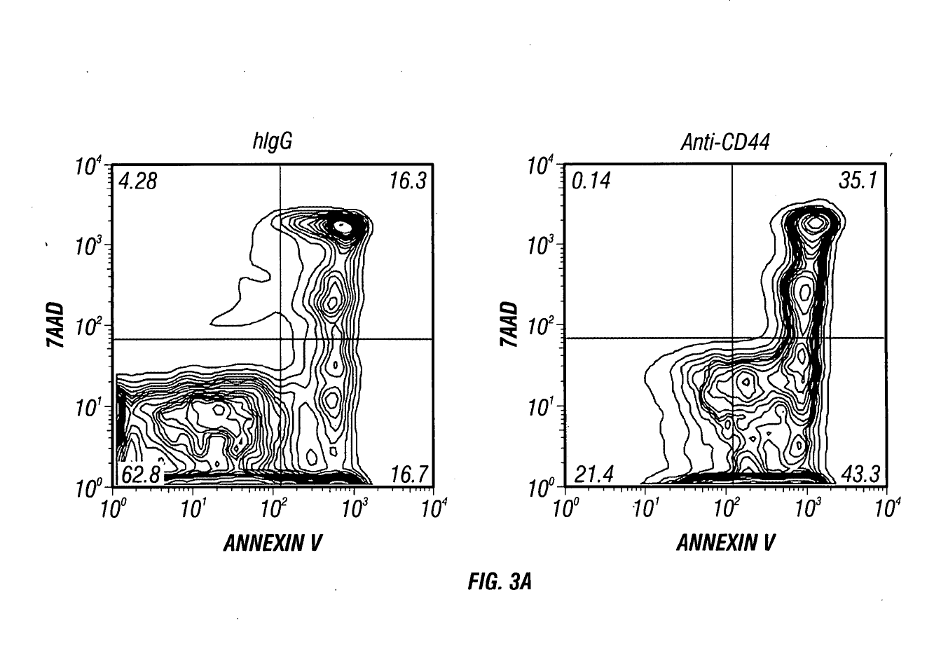

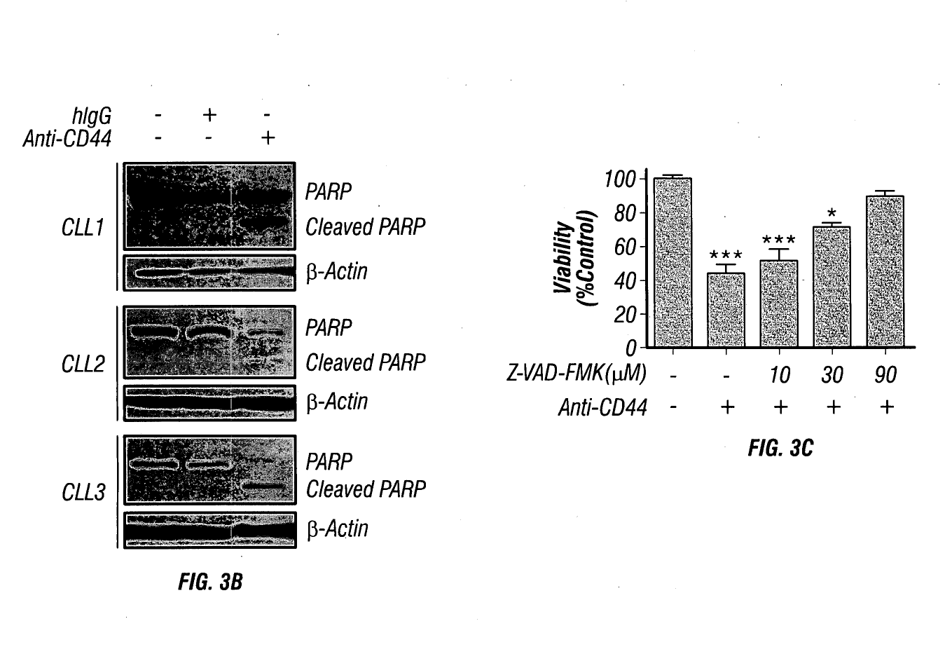

As depicted in FIG. 3, CLL B cells were cultured for 48 hours in the presence of 50 ug/ml anti-CD44 mAb or hIgG control Ab. FIG. 3A depicts cells stained with Annexin V and 7AAD and analyzed by flow cytometer. The relative fluorescence intensities are depicted on the X and Y axis respectively. Cells in the lower and upper right quadrant are apoptotic cells. In FIG. 3B, cell lysates were harvested and analyzed by Western blot for the cleaved fragment of PARP. β-actin was used as loading control. (C) Cells were cultured with anti-CD44 mAb in the absence or presence of a pan-caspase inhibitor, Z-VAD-FMK, at various concentrations for 48 hours. The cells were analyzed by flow cytometer following DIOC6 and PI staining. Data were compared to samples without treatment as 100% viability. Results shown are mean±SEM from three different CLL samples. Statistical significance was determined using Dunnett's multiple comparison test. * indicates P<0.05, ** indicates P<0.01, *** indicates P<0.001.

At least 2-fold increase of the annexin V positive apoptotic cells was found in CLL cells treated with RG7356 compared to that with control hIgG (FIG. 3A). Induction of PARP cleavage was also detected in these RG7356 treated samples (FIG. 3B). Finally, cell death was rescued by a pan-caspase inhibitor, Z-VAD-FMK, at a dose-dependent fashion (FIG. 3C), indicating RG7356 induced apoptosis is caspase-dependent in CLL cells.

Influence of CLL Microenvironment.

Cells of the microenviroment have been reported to protect CLL cells from spontaneous and drug-induced apoptosis (Burger et al. Blood 2009, 114:3367-3375; Meads et al. Nat Rev Cancer 2009, 9:665-674). To study the impact of CLL microenvironment on RG7356 induced apoptosis, cell viability assays were carried out on CLL cells co-cultured with patient marrow-derived MSCs.

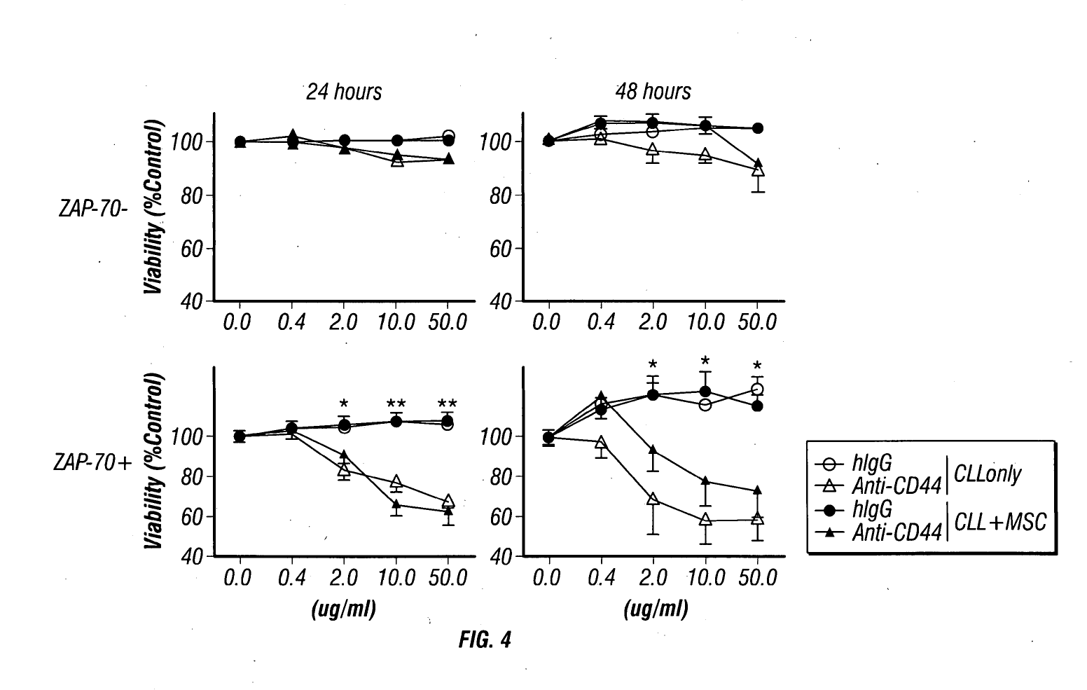

FIG. 4 depicts CLL cells cultured either alone or in the presence of mesenchymal stromal cells (MSC) were incubated with anti-CD44 mAb or hIgG control antibody at the indicated concentration for 24 or 48 hours. Viability was measured by staining and flow cytometry. Data were normalized to the population of PI^{neg}/DiOC₆^{hi} at time point 0 as 100% viability. Results shown are mean+/−SEM from 3 different patients of each group. * indicates a statistically significant difference between anti-CD44 mAb treatment and hIgG treatment (Paired Student's t test).

Consistent with prior observations, RG7356 treated ZAP-70+ CLL cells showed a rapid and significant reduction in viability regardless the presence of MSCs, and approximately 50% of cells were dead by 48 hours (FIG. 4). In contrast, ZAP-70− CLL cells were not affected regardless the presence of MSCs.

Anti-CD44 mAb (RG7356) Blocks Hyaluronic Acid (HA)-Induced Signaling and Survival of ZAP-70+ CLL Cells In Vitro.

Because MSCs derived from healthy individuals as well as MSC derived from patients with multiple myeloma have been shown to express HA synthases as well as HA, (Calabro et al. Blood 2002, 100:2578-2585; Jung et al. Biochem Biophys Res Commun 2011, 404:463-469) a principle ligand of CD44, we investigated the effect of HA on CLL cell survival and signaling. CLL cells were cultured for 24 hours in the absence or presence of 50 μg/ml HA and cell viability was assessed using DiOC₆/PI staining with flow cytometry analysis.

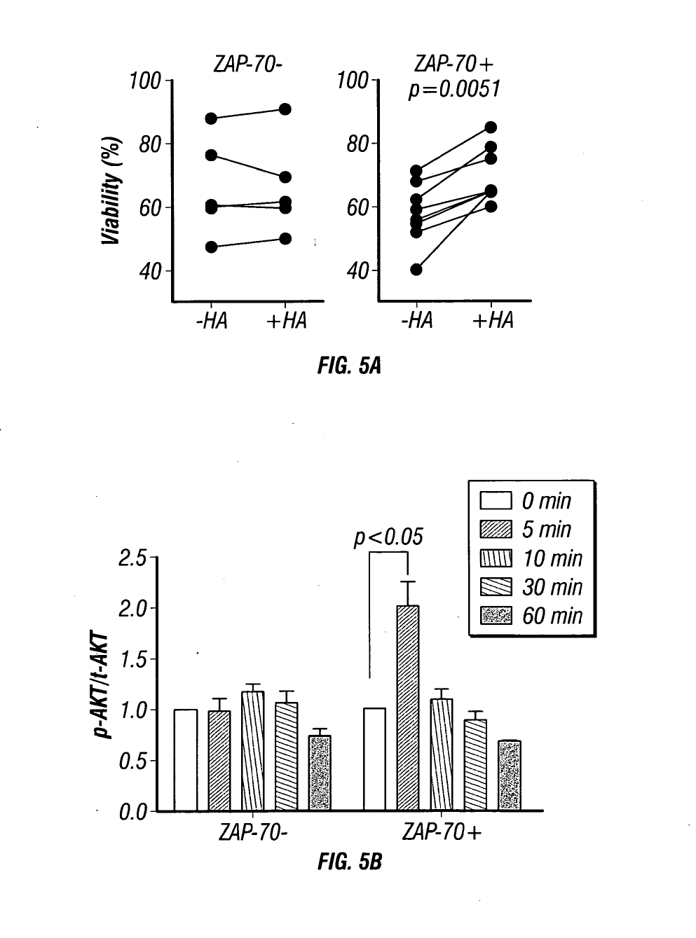

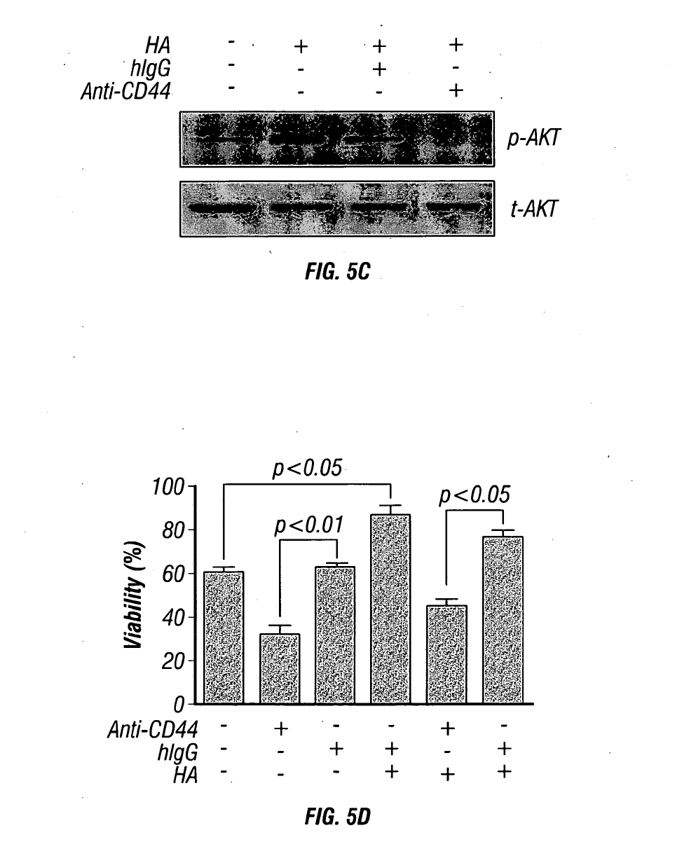

FIG. 5A depicts purified CLL cells from ZAP-70^{neg} CLL samples (n=5) or ZAP-70⁺ CLL samples (n=7) incubated with or without HA (50 ug/ml) for 24 hours. CLL cells were harvested and stained with DiOC6/PI and analyzed by flow cytometry. The data shown depicts the percent of DiOC₆+PI− viable cells for each patient tested. FIG. 5B depicts cell lysates harvested at different time points from CLL samples (n=3, ZAP-70^{neg} or ZAP-70⁺) stimulated with HA (50 ug/ml) and analyzed by a phosphorylation AKT (p-AKT)/total AKT (t-AKT) specific ELISA assay. Results shown are mean+/−SD of the level of p-AKT normalized to t-AKT at different time points relative to that of pre-treatment (0 min). P<0.05 indicates statistical significance of differences analyzed using paired student's t-test. In FIG. 5C, ZAP-70⁺ CLL samples were pretreated with or without anti-CD44 mAb (50 ug/ml) for 20 minutes, then stimulate with HA (50 ug/ml) for 5 minutes. Cells were lysed and analyzed by western blot for the expression of p-AKT and t-AKT. In FIG. 5D, ZAP-70⁺ CLL samples were incubated with or without Anti-CD44 mAb and with or without HA for 24 hours. Cells were harvested and analyzed by flow cytometry following DiOC6/PI staining. The percent of DiOC6⁺/PI^{neg} viable cells was shown. Statistical significance was determined by One-way ANOVA following Tukey's multiple comparison test.

A low to moderate increase in cell viability was observed in ZAP-70+ patient samples treated with HA (FIG. 5A, right panel). In contrast, HA treatment had little or no effect on the viability of most ZAP-70− cases. When examined the possible signaling pathways that can be induced by HA in CLL cells, we found that phosphorylation of AKT was rapidly increased between 5 to 10 minutes after HA treatment using a pAKT/total AKT specific ELISA assay (FIG. 5B). Consistent with the viability result, phosphorylation of AKT signaling is preferentially induced by HA in ZAP-70+ cases, although all the samples tested had similar levels of CD44 expression (data not shown). To evaluate the effect of RG7356 on HA-induced signaling and cell survival, ZAP-70+ CLL cells were pretreated with RG7356 and then stimulated with HA. Induction of either p-AKT signaling or survival after HA treatment was abolished (FIG. 5C-D), suggesting that RG7356 is very effective in blocking interactions between CD44 and HA, which can be accumulated in the microenvironment.

RG7356 Abrogates ZAP-70 Downstream BCR Signaling Cascade.

To further investigate the mechanism by which RG7356 inhibits CLL cell survival, we performed internalization assay.

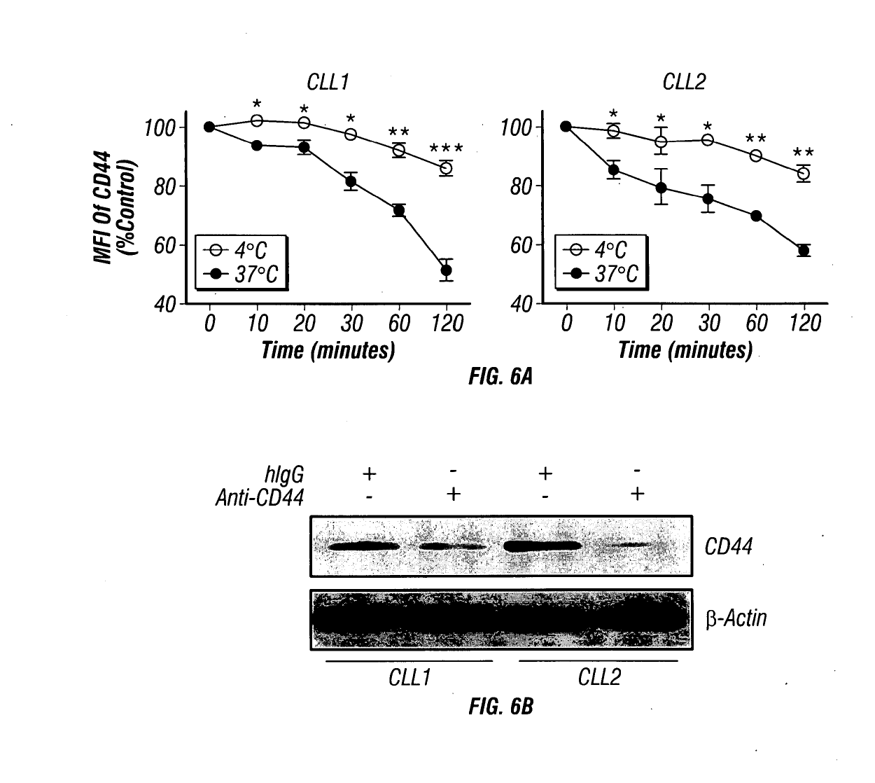

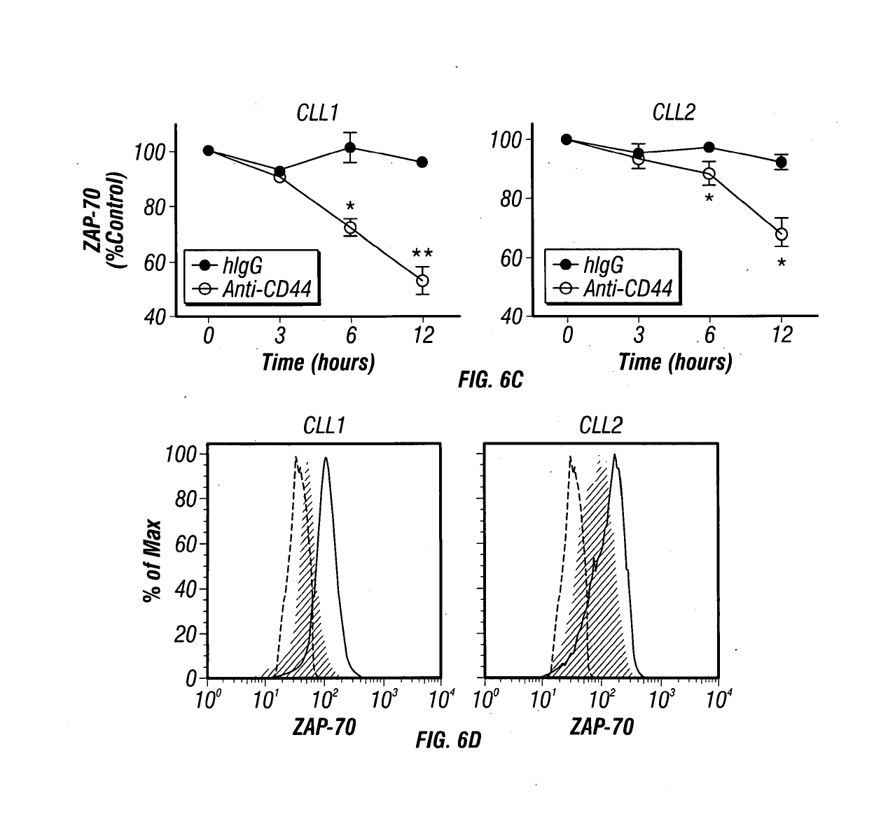

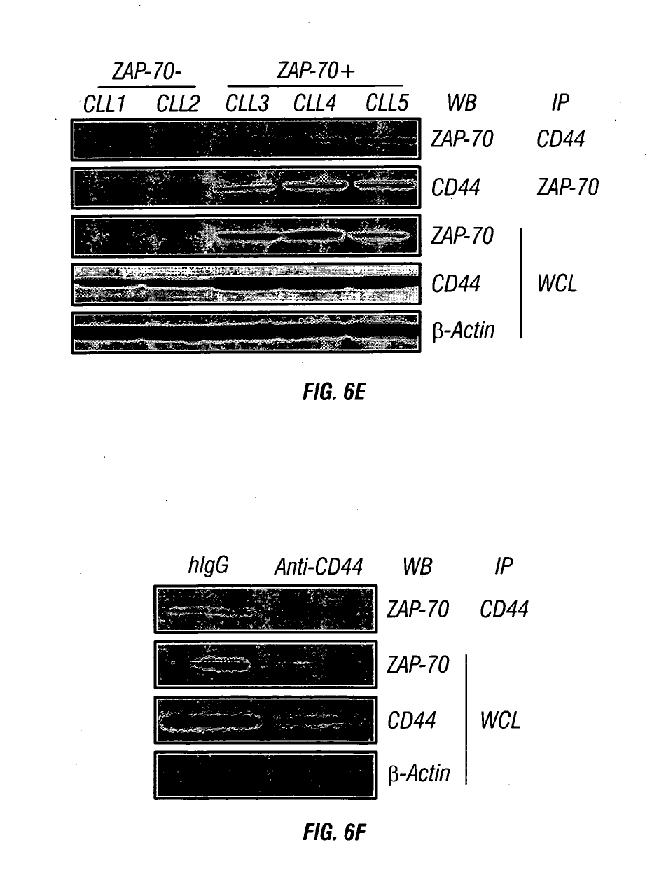

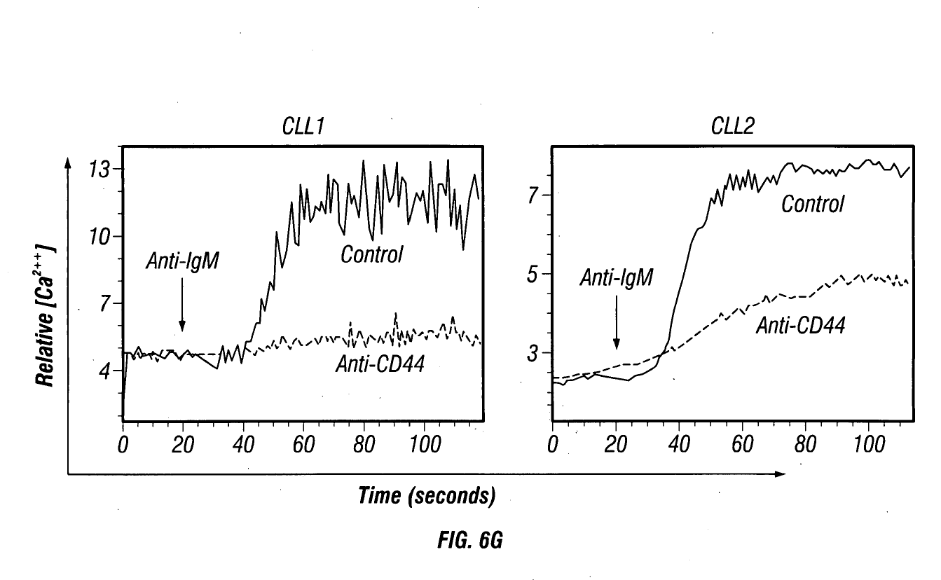

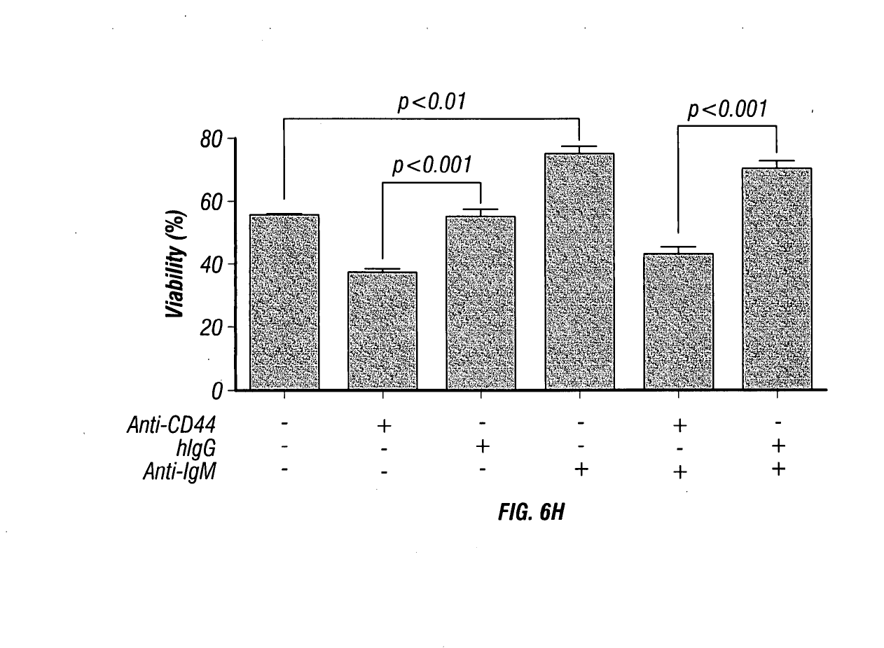

FIG. 6A depicts internalization of anti-CD44 mAb by CLL cells. CLL cells were stained with Alexa-647 conjugated anti-CD44 mAb, incubated at either 4° C. or 37° C., and analyzed at indicated time points by flow cytometry. Data were presented as CD44 mean fluorescence intensity (MFI) normalized to that from cells at time 0 as 100%. FIG. 6B depicts reduction of CD44 protein levels following CD44 mAb treatment. CLL samples were treated with either hIgG control Ab or Anti-CD44 mAb (50 ug/ml) for 48 hours, and cell lysates were analyzed by immunoblot. FIG. 6D depicts decreased ZAP-70 protein levels following CD44 mAb treatment. CLL cells were incubated with anti-CD44 mAb or hIgG control Ab for indicated time period, stained with Alexa-488-conjugated anti-ZAP-70 antibody, and analyzed by flow cytometry. Shown are 2 representative CLL samples that were ZAP-70⁺. In FIG. 6D, representative histograms depict the fluorescence intensities of ZAP-70 in two Zap-70+ CLL samples (CLL1 and CLL2) treated with anti-CD44 mAb (open) or IgG control (shaded) for 48 hours. The level of ZAP-70 in CD19+ normal B cells serves as negative control (open histograms with dashed line). FIG. 6E demonstrates that CD44 physically associates with Zap-70 in CLL cells. Protein lysates of CLL cells from different patients were immunoprecipitated (IP) with either anti-CD44 mAb or anti-ZAP-70 Ab. The bound products or whole cells lysates (WCL) were probed by immunoblot using the antibodies indicated in the WB column. FIG. 6F depicts the down modulation of CD44 and ZAP-70 protein complex following anti-CD44 mAb treatment. ZAP-70⁺ CLL cells were treated with anti-CD44 mAb (50 ug/ml) or hIgG control Ab, protein lysates immunoprecipitated (IP) with anti-CD44 mAb and subjected to immunoblotting with the indicated antibodies. FIG. 6G demonstrates that treatment of anti-CD44 mAb reduces IgM-induced calcium flux in CLL cells. Zap-70⁺ CLL samples were first labeled with the fluorescent calcium-indicator Fluo-4AM, preincubated with either anti-CD44 mAb (50 ug/ml) or hIgG control Ab for 12 hours, then stimulated with anti-IgM, and the fluorescence intensity was recorded over time by FACS. The lines represent the changes in fluorescence intensity (on the y axis) over time (x axis) for control CLL cells (black line) or cells preincubated with anti-CD44 mAb (gray line). Arrow indicates the time when anti-IgM was added. FIG. 6H shows that anti-CD44 mAb mitigates IgM-induced survival in CLL cells. ZAP-70⁺ CLL samples were incubated with or without 50 ug/ml anti-CD44 mAb or IgG control Ab in the presence or absence of anti-IgM (10 ug/ml) for 48 hours. Cells were harvested, stained with DiOC₆/PI and analyzed by flow cytometry for viability measurement. A representative data was shown from one of the three patient samples tested. Each bar depicts the mean proportion of DiOC₆^{hi}/PI^{neg} viable cells from triplicates. Error bar indicates SEM. * indicates statistical significance of differences analyzed using One-way ANOVA following Tukey's multiple comparison test.

Upon binding to Alex647-conjugated RG7356, rapid internalization of cell surface CD44 was detected 10 minutes after 37° C. incubation and more than 40% mean fluorescence intensity (MFI) reduction displayed by 2 hours (FIG. 6A). No reduction of surface IgM was observed regardless of treatments for up to 24 hours (data not shown), demonstrating that RG7356 is specific to CD44. Western blot analysis of the total CD44 protein level in RG7356 treated CLL cells also revealed a significant reduction (FIG. 6B). Interestingly, ZAP-70 expression level was also reduced significantly after 6 hours of RG7356 treatment and by 12 hours ZAP-70 level was reduced at least 30-50% (FIG. 6C-D). Results from immunoprecipatation analysis demonstrated that ZAP-70 and CD44 formed a complex in all the ZAP-70+ CLL cells tested but not in ZAP-70− cases (FIG. 6E), suggesting that ZAP-70 is probably involved in CD44 survival signaling in CLL cells. Indeed, treatment with RG7356 disrupted the ZAP-70/CD44 complex as shown in FIG. 6F. Subsequently, BCR downstream signaling, e.g., intracellular calcium flux, was also inhibited (FIG. 6G). In addition, pro-survival effect of anti-IgM stimulation was abrogated by RG7356 treatment compared to that with control antibody (FIG. 611).

RG7356 Impairs CLL Cell Survival in a Xenograft Animal Model.

To evaluate the in vivo efficacy of RG7356, we established a xenograft CLL parking animal model using highly immunodeficient RAG2/gamma chain knockout mice (Rag2^{-/-}γc^{-/-}) which allows us to determine the residual CLL cells quantitatively.

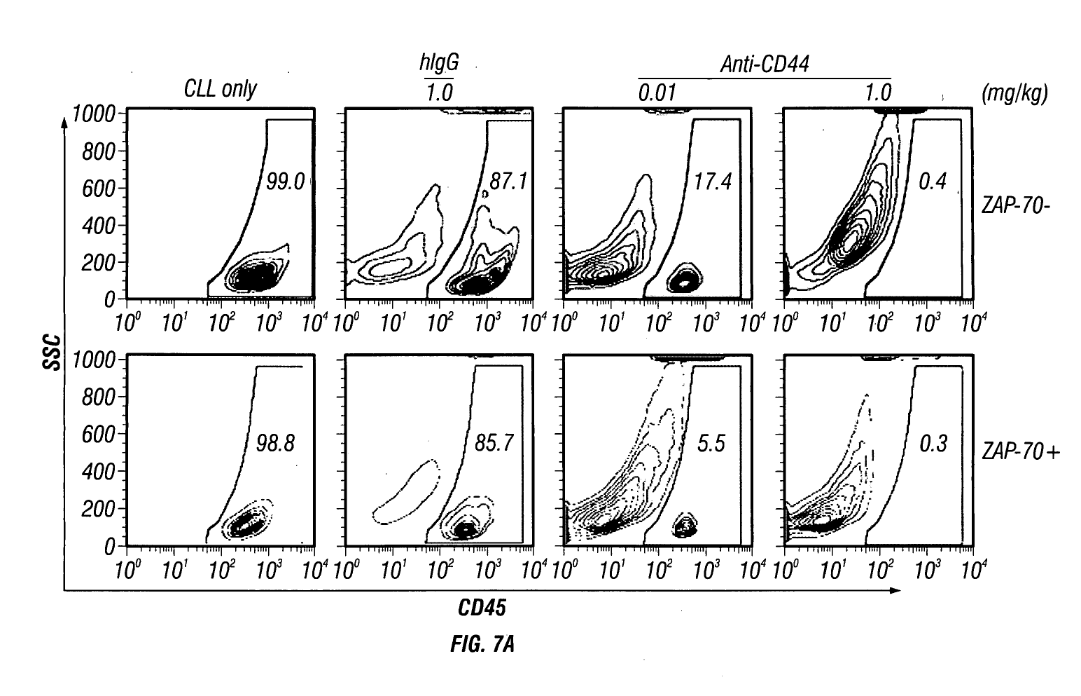

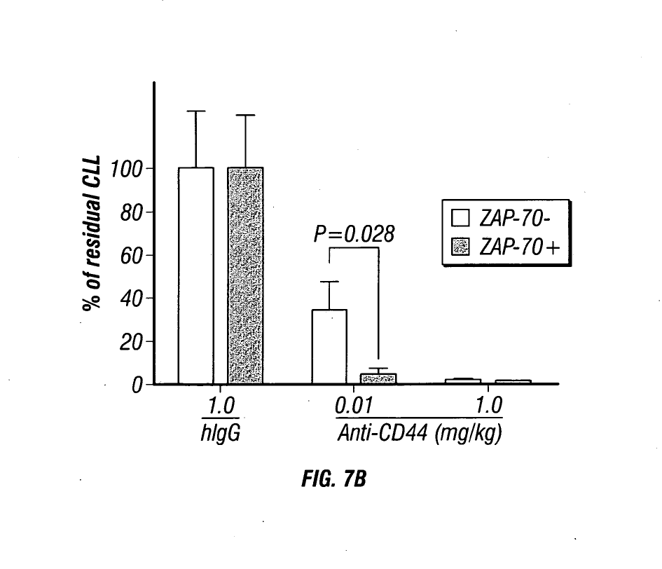

As depicted in FIG. 7, CLL cells were injected to the peritoneal cavity of Rag2^{-/-}γc^{-/-} mice one day prior to mAb injection. Peritoneal lavage were collected 7 days after cell injection and subjected to residual CLL determination by cell counting and FACS analysis following human-specific CD5, CD19 and CD45 staining. FIG. 7A shows contour plots of two representative CLL samples treated with mAbs at either low or high doses. Cells on the lower right gates are human CLL cells, and those numbers were used to generate the bar graph shown in FIG. 7B. Each bar in the graph of FIG. 7B represents percentage of residual CLL cells harvested form mice after anti-CD44 mAb treatment at different concentrations and normalized to that from mice treated with control hIgG as 100%. Data shown are mean+/−SEM from 3 different patients with n=3 in each group. P indicates a statistically significant difference between anti-CD44 mAb treatment and hIgG treatment, as calculated by Student's t test.

Dose titration study supported our previous observations that ZAP-70 CLL cells are more responsive to RG7356 treatment than ZAP-70− cells at a single low dose as little as 0.01 mg/Kg body weight (FIG. 7A). Nevertheless, more than 90% of CLL cells regardless of ZAP-70 expression levels were cleared from mice treated with 1 mg/Kg of RG7356 compared to that with control hIgG as 100% recovery (FIG. 7B), suggesting that RG7356 is highly efficient in clearing CLL cells in a niche dependent manner regardless of patients' disease characteristics.

Mode of Action of RG7356 is Antibody-Dependent Cell Phagocytosis (ADCP).

Although Rag2^{-/-}γc^{-/-} mice are deficient in B, T and NK cells, residual macrophages are still present in the peritoneal cavity. To further evaluate the role of peritoneal macrophages in RG7356-mediated cytotoxicity in ZAP-70− CLL cells, cells were treated in the presence or absence of thioglycolate enriched peritoneal mouse macrophages and subjected to phagocytosis and cell viability assays.

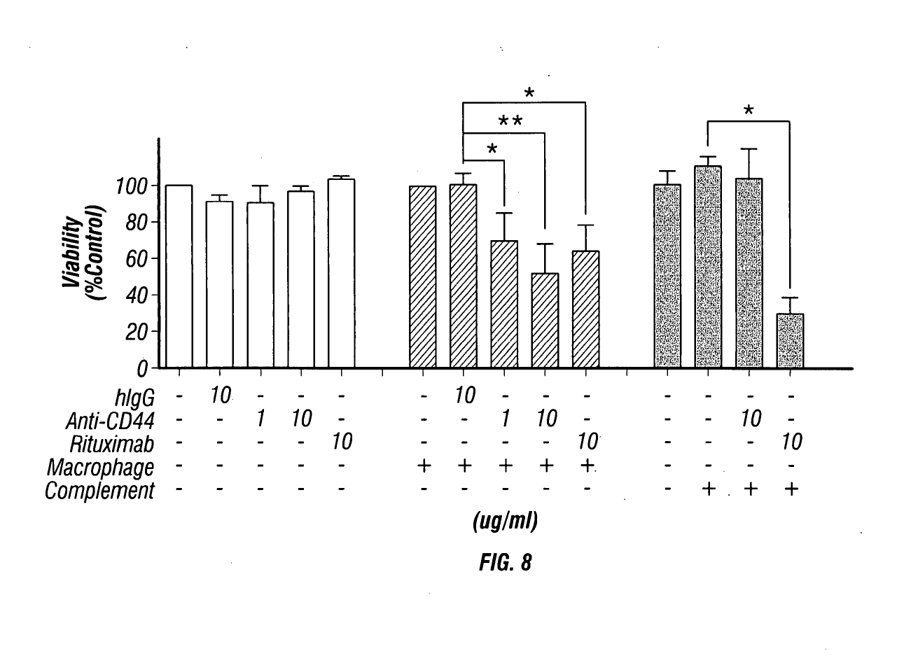

FIG. 8 depicts CLL samples preincubated with anti-CD44 mAb, control hIgG Ab, or Rituximab at indicated concentration for 30 minutes on ice. The cells were further incubated at 37° C. either alone (open bars) or in the presence of macrophages (grey bars) at 1:5 ratio or rabbit complement (right) for 3 more hours. Cells were collected, stained and analyzed for viable CLL cells (PI^{neg}gDiOC₆^{hi}) by flow cytometry. Data shown are mean+/−SEM from 5 different patients per group and normalized to the corresponding control samples as 100% viability. * indicates P<0.05, ** indicates P<0.01 (One-way ANOVA following Tukey's multiple comparison test).

Cell viability was markedly reduced by about 50% after 3 hours incubation with either RG7356 or Rituximab only in cells co-cultured with peritoneal macrophages (FIG. 8, grey bars) but not in cells alone (open bars), suggesting an active phagocytosis occurred in the system. However, no complement-mediated cytotoxicity was observed in RG7356-treated cells compared to ones treated with Rituximab as positive control (FIG. 8, filled bars).

Viability of CLL cells treated with anti-CD44 mAb or Rituximab

High level expression of surface CD44 protein in CLL cells encouraged us to evaluate the cytotoxic activity against CLL cells of a newly developed, humanized anti-CD44 mAb (RG7356).

In FIG. 9, each dot on the left graph represents the expression level of CD44 (MFIR) by B cells from an individual patient with CLL, gated on CD19+CD5+ B cells. The line indicates the median CD44 expression level by each group. Each dot on the right graph represents the relative viability of cells from one patient cultured with 50 ug/ml anti-CD44 mAb for 24 hours. The percent viable cells have been normalized to the viability of control mAb treated cells. The line indicates the median or viability of cells treated with anti-CD44 mAb by the group. Statistical significance was determined using Student's t test.

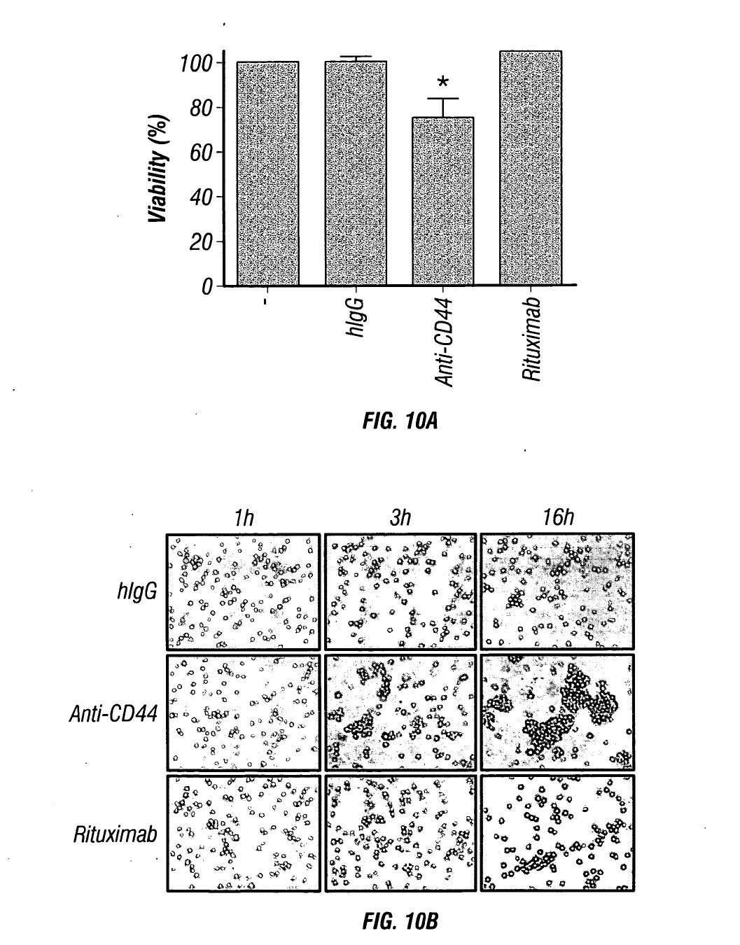

As low as 10 ug/ml RG7356 shows great direct cell killing effect, which was clearly superior compared with rituximab at similar dose that does not induce proapototic activity (FIG. 10A). However, little or no effect on survival of normal B cells treated with this RG7356 at high dose was observed, suggesting this anti-CD44 mAb is relatively safe to normal B cells. Moreover, apoptotic cell killing by anti-CD44 mAb does not require IgG cross-linking and thus the mode of direct cell death induction induced by anti-CD44 mAb, resembles that of so-called type II CD20 mAbs.

Type II CD20 mAbs are characterized by reduced ability to mediate CDC activity compared with type I mAbs, but have been demonstrated to outperform type I mAbs with respect to their B cell-depleting activity. (Mossner et al. Blood 2010, 115:4393-4402; Beers et al. Blood 2008, 112:4170-4177) In this context, it is interesting to note that anti-CD44 mAb likewise lacks CDC activity.

FIG. 10A depicts CLL samples (n=4) incubated with Anti-CD44 mAb, rituximab or control antibody (10 ug/ml) for 24 hours. CLL cells were harvested and stained with DiOC6/PI and analyzed by flow cytometry. The data shown depicts the percent of DiOC6+PI− viable cells. P indicates statistical significance of differences analyzed using Dunnett's multiple comparison test. FIG. 10B depicts the phenotypic analysis of mAb treatment on CLL cells. Primary CLL cells were incubated with anti-CD44, rituximab or human IgG control antibody at a concentration of 50 ug/ml at 37° C. in cell culture medium and photomicrographs were recorded at the indicated time points (20× phase-contrast objective).

Rapid homotypic aggregation after incubation with anti-CD44 mAb (FIG. 10B) provide evidence that anti-CD44 mAb can be considered to be a type II mAbs and is associated with highly effective direct cell killing. (Alduaij et al. Blood 2011, 117:4519-4529)

What is claimed is:

1. An isolated antibody or antibody fragment which specifically binds to CD44.

2. The antibody or antibody fragment of claim 1, wherein that antibody fragment is selected from the group consisting of a Fab fragment, a F(ab)2 fragment, an FV fragment, a single chain FV (scFV) fragment, a dsFV fragment, a CH fragment and a dimeric scFV.

3. The antibody or antibody fragment of claim 1, wherein the antibody or antibody fragment is humanized.

4. The antibody or antibody fragment of claim 1, wherein the antibody or antibody fragment specifically binds to CD44 expressed on CLL cells.

5. A pharmaceutical composition comprising the antibody or antibody fragment of claim 1 and a pharmaceutically acceptable carrier

6. An isolated nucleic acid molecule which encodes the antibody of claim 1.

7. An expression vector which contains the nucleic acid molecule of claim 6.

8. A method of producing the antibody or antibody fragment of claim 1, the method comprising:
i) transforming a host cell with an expression construct comprising,
a nucleic acid molecule encoding the antibody or antibody fragment of claim 1; and
ii) culturing the host cell under conditions suitable for producing the conjugate, thereby producing the protein conjugate.

9. A method for detecting CD44 protein in a sample, the method comprising:
(a) contacting the sample with a detectably labeled antibody or antibody fragment of claim 1; and
(b) detecting immunoreactivity between the detectably labeled peptide and CD44 in the sample.

10. The method of claim 9, further comprising (c) determining whether an increase or decrease in an amount of CD44 protein has occurred in the subject in comparison to a control level of CD44.

11. A method of targeting an antibody or antibody fragment to a cell having an CD44 receptor, the method comprising contacting the cell with an antibody of the invention.

12. A kit to detect the presence of CD44 protein in a sample from a subject that is known or suspected to contain hematological malignant cells, comprising the antibody or antibody fragment of claim 1 and instructions for its use in an assay environment.

13. A method for treating or preventing a hematological malignancy, the method comprising administering to the subject in need thereof a therapeutically effective amount an antibody to CD44.

14. A method of monitoring a therapeutic regimen for treating a subject having or at risk of having an hematological malignancy, comprising determining a change in activity or expression of CD44 protein as a result of administering an antibody specific for CD44, thereby monitoring the therapeutic regimen in the subject.

15. A method for treating or preventing CLL in which an anti-CD44 antibody binding to CD44 on CLL cells confers a survival advantage thereon, comprising administering the antibody or antibody fragment of claim 1.

16. A method for treating or preventing a hematological malignancy in a subject, the method comprising administering to a subject in need thereof a therapeutically effective amount of an antibody that specifically binds CD44, wherein the hematological malignancy is refractory to chemotherapy and/or biotherapy.

17. The method of claim 16, wherein the chemotherapy comprises a purine nucleoside analog.

18. The method of claim 16, wherein the chemotherapy comprises an alkylating agent.

19. The method of claim 16, wherein the chemotherapy comprises a purine nucleoside analog and an alkylating agent.

20. The method of claim 16, wherein the hematological malignancy is refractory to chemotherapy and biotherapy.

21. The method of claim 16 or 20, wherein the biotherapy comprises a monoclonal antibody.

22. The method of claim 21, wherein the monoclonal antibody is an anti-CD20 antibody.

23. The method of claim 16, wherein the hematological malignancy is leukemia.

24. The method of claim 23, wherein the leukemia is lymphocytic leukemia.

25. The method of claim 24, wherein the lymphocytic leukemia is B-cell chronic lymphocytic leukemia (CLL).
